SEQUENCE LISTING (7 sequences)

SEQ ID NO: 1 - protein, 6 aa, synthetic construct
  NON_TER 1: note = N-terminal CH3(CH2)14-CO- modification
VVAAEE

SEQ ID NO: 2 - protein, 5 aa, synthetic construct
  NON_TER 1: note = N-terminal CH3(CH2)14-CO- modification
VAAEE

SEQ ID NO: 3 - protein, 6 aa, synthetic construct
  NON_TER 1: note = N-terminal CH3(CH2)14-CO- modification
AAVVEE

SEQ ID NO: 4 - protein, 6 aa, synthetic construct
  NON_TER 1: note = N-terminal CH3(CH2)8-CO- modification
VVAAEE

SEQ ID NO: 5 - protein, 5 aa, synthetic construct
  NON_TER 1: note = N-terminal CH3(CH2)8-CO- modification
VAAEE

SEQ ID NO: 6 - protein, 29 aa, synthetic construct
  NON_STD 16: note = X is 2-aminoisobutyric acid (Aib)
HSQGTFTSDYSKYLDXARAEEFVKWLEST

SEQ ID NO: 7 - protein, 29 aa, synthetic construct
  NON_STD 16: note = X is 2-aminoisobutyric acid (Aib)
  NON_STD 25: note = methoxycoumarin-modified lysine at position 25
HSQGTFTSDYSKYLDXARAEEFVKKLEST

(19) United States
(12) Patent Application Publication
(10) Pub. No.: US 2023/0357349 A1
(43) Pub. Date: Nov. 9, 2023

(54) GLUCAGON DELIVERY VIA ENZYMATIC ACTUATION

(71) Applicant: University of Notre Dame du Lac, South Bend, IN (US)

(72) Inventors: Matthew J. Webber, South Bend, IN (US); Sihan Yu, South Bend, IN (US)

(21) Appl. No.: 18/310,733

(22) Filed: May 2, 2023

Related U.S. Application Data
(60) Provisional application No. 63/364,047, filed on May 3, 2022.

(51) Int. Cl. C07K 14/605; A61P 3/10

(52) U.S. Cl. CPC C07K 14/605; A61P 3/10; A61K 38/00

(57) ABSTRACT

Described herein are glucose-stabilized materials for glucose-responsive delivery of glucagon or a glucagon analogue to combat hypoglycemia and related disorders. Exemplary glucose-stabilized materials of the present invention include hydrogels comprising glucagon or a glucagon analogue and a peptide. Enzymatic control of molecular self-assembly and hydrogelation described herein enables encapsulation and glucose-responsive delivery of a therapeutic to address low glucose.

### CROSS-REFERENCE TO RELATED APPLICATIONS

This application claims priority to U.S. Provisional Patent Application No. 63/364,047, filed on May 3, 2022, which is incorporated by reference herein in its entirety.

### FEDERALLY SPONSORED RESEARCH

This invention was made with government support under grant number 1944875 awarded by the National Science Foundation. The government has certain rights in the invention.

### REFERENCE TO SEQUENCE LISTING

This application was filed with a Sequence Listing XML in ST.26 XML format accordance with 37 C.F.R. § 1.831. The Sequence Listing XML file submitted in the USPTO Patent Center, “092012-0012-US02-Sequence_listing_xml_4 Apr. 2023.xml,” was created on Apr. 4, 2023, contains 7 sequences, has a file size of 9.91 Kbytes, and is incorporated by reference in its entirety into the specification.

### BACKGROUND

Stimuli-responsive strategies have been a topic of extensive focus towards the creation of materials for more precise drug delivery. The use of disease-relevant proteins or analytes to prompt drug release from a circulating nanocarrier or localized depot affords opportunities for improved spatiotemporal control of therapeutic action. Blood glucose management in diabetes presents one circumstance where temporal control of therapeutic bioavailability is particularly relevant. In pursuit of high-fidelity blood glucose control, most approaches have focused on engineering glucose-responsive release and/or insulin activity to replace its deficient or defective signaling and combat hyperglycemia. However, the clinical use of insulin poses substantial risk of overdose. The average person with diabetes has 1-2 serious hypoglycemic episodes each year; most such episodes are attributed to excessive insulin activity. Severe hypoglycemia at night is especially common in children and can prove lethal if not corrected quickly, thus referred to as “dead-in-bed” syndrome. Accordingly, insulin is dosed conservatively and even underdosed in many cases, thereby preferencing chronic health complications from blood glucose instability and hyperglycemia in order to avoid the acute risks of hypoglycemia. Glucagon functions in the healthy endocrine system as an antagonist to insulin by raising blood glucose upon hypoglycemia and, as such, is an interesting therapeutic target. Integrating glucose-responsive strategies for glucagon delivery may afford enhanced precision in insulin-centered blood glucose control while mitigating the severe risks of hypoglycemia.

The construction of biomaterials and drug delivery devices from supramolecular interactions offers routes to endow stimuli-responsivity using tunable non-covalent associations. In the field of supramolecular materials, non-equilibrium systems that form transiently by input of energy or consumption of chemical fuels constitute an exciting and growing body of research. Enzymes are useful components of many non-equilibrium and/or fueled systems reported so far due to their ability to chemically transform a pre-assembled molecule or promote an environment favoring its (transient) assembly. The use of disease-relevant enzymes or their substrates to facilitate transformations in supramolecular materials offers a possible strategy for improved therapeutic precision in drug delivery.

In the context of glucose-responsive materials, glucose oxidase (GOₓ) has been used to actuate glucose levels into a material-directing stimulus. GOₓ catalyzes the conversion of one molecule of D-glucose into glucono-δ-lactone and H₂O₂, with the former hydrolyzing to gluconic acid. Glucose-responsive hydrogels that incorporate GOₓ sensing use the reduction in microenvironmental pH from gluconic acid production to drive swelling or bond rupture in a polymeric network. GOₓ has also been used to regulate gelation in pH-sensitive peptide gelators.

What are needed are compositions and methods for actuating glucose oxidase (GOₓ) activity.

### SUMMARY

One embodiment described herein is a composition comprising a peptide of formula (I):

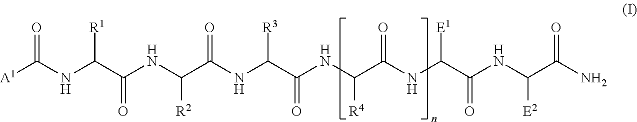

wherein:
A¹ is C₆₋₂₀alkyl;
R¹, R², R³, and R⁴, at each occurrence, are independently C₁₋₆alkyl, C₃₋₆cycloalkyl, C₁₋₂haloalkyl, C₁₋₄hydroxyalkyl, halogen, —CN, —OR¹¹, —NHR¹¹, —CO₂R¹¹, —N(R¹¹)₂, —C(O)NHR¹¹, or —C(O)N(R¹¹)₂;
R¹¹, at each occurrence, is independently hydrogen, C₁₋₄alkyl, or C₃₋₆cycloalkyl;
n is 1-3; and
E¹ and E² are each independently

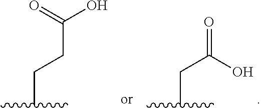 [SMILES: *CCC(=O)O ; *CC(=O)O]

In another aspect, A¹ is C₉₋₁₅alkyl. In another aspect, A¹ is linear. In another aspect, R¹, R², R³, and R⁴, at each occurrence, are each independently C₁₋₄alkyl. In another aspect, R¹, R², R³, and R⁴, at each occurrence, are each independently methyl or isopropyl.

In another aspect,

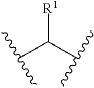

is

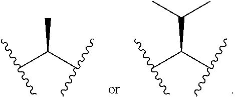

In another aspect,

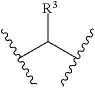

is

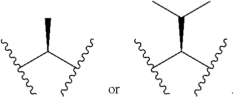

In another aspect,

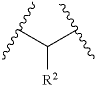

is

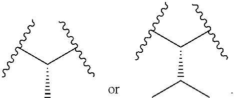

In another aspect,

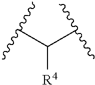

is

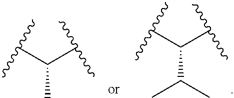

In another aspect, E¹ and E² are each

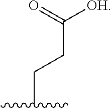 [SMILES: *CCC(=O)O]

In another aspect,

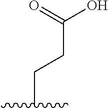 [SMILES: *CCC(=O)O]

is

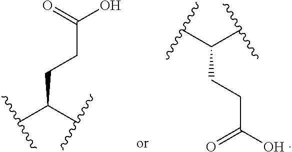

In another aspect, the peptide of formula (I) is a peptide of formula (I-a):

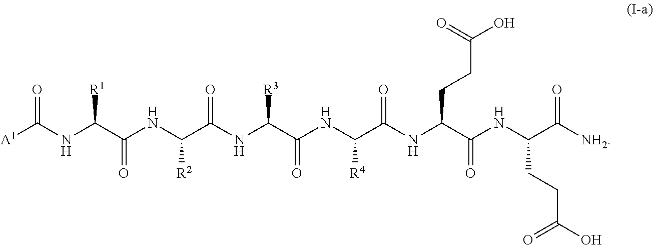

In another aspect, the peptide of formula (I) is a peptide selected from the group consisting of:

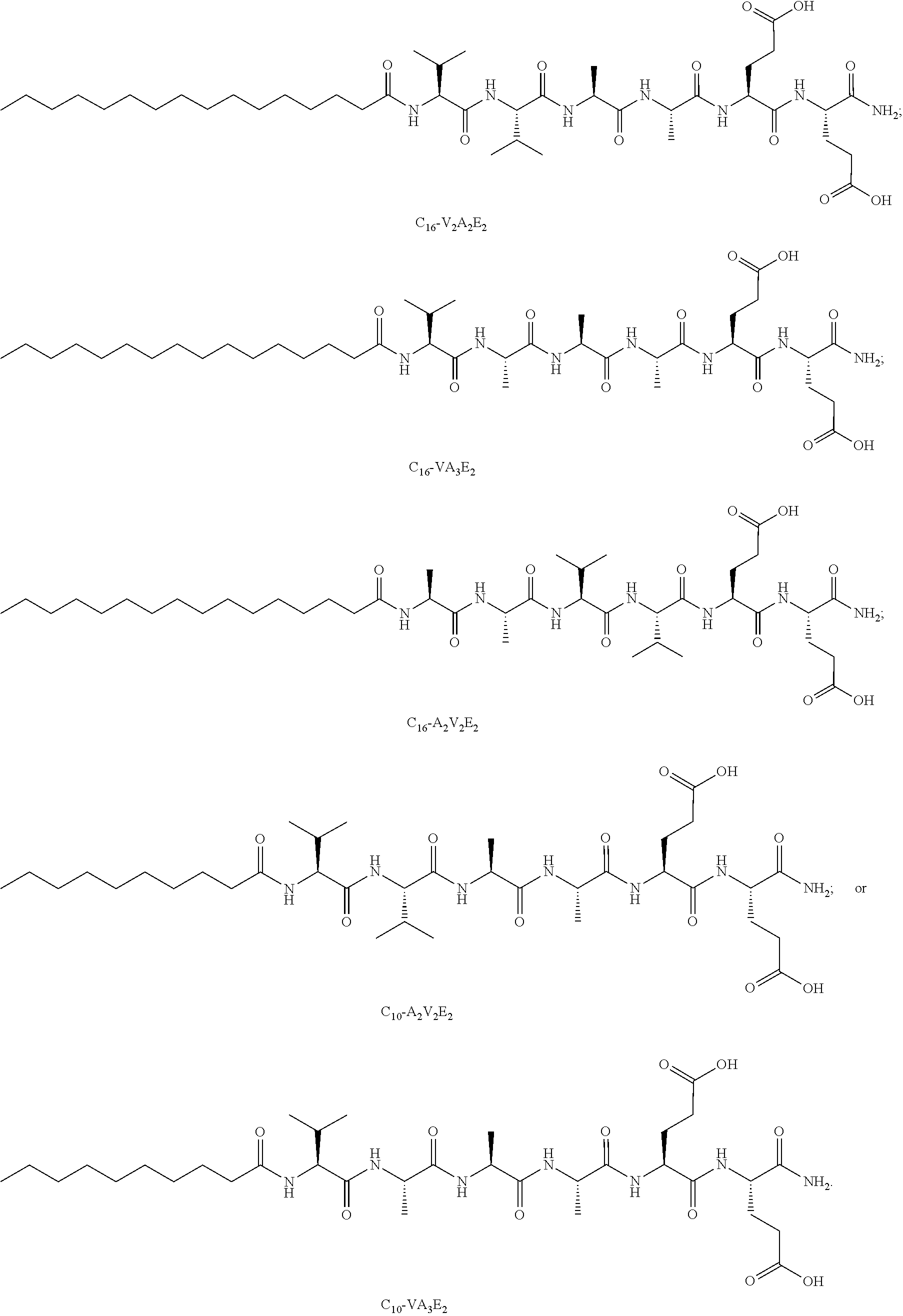

In another aspect, the peptide of formula (I) is:

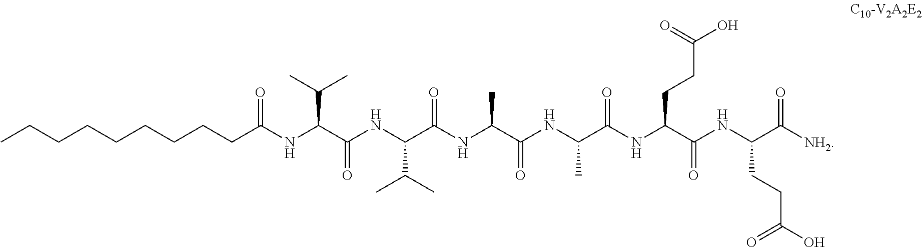 [SMILES: CCCCCCCCCC(=O)N[C@H](C(=O)N[C@H](C(=O)N[C@@H](C)C(=O)N[C@@H](C)C(=O)N[C@@H](CCC(=O)O)C(=O)N[C@@H](CCC(=O)O)C(N)=O)C(C)C)C(C)C]

In another aspect, at a pH of about 5, the peptide of formula (I) self-assembles to form a hydrogel.
In another aspect, at a pH of about 7, the hydrogel disassembles.

Another embodiment described herein is a hydrogel comprising:
glucagon or a glucagon analogue;
a peptide of formula (I):

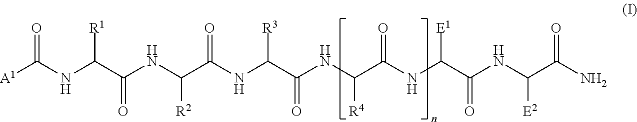

wherein:
A¹ is C₆₋₂₀alkyl;
R¹, R², R³, and R⁴, at each occurrence, are independently C₁₋₆alkyl, C₃₋₆cycloalkyl, C₁₋₂haloalkyl, C₁₋₄hydroxyalkyl, halogen, —CN, —OR¹¹, —NHR¹¹, —CO₂R¹¹, —N(R¹¹)₂, —C(O)NHR¹¹, or —C(O)N(R¹¹)₂;
R¹¹, at each occurrence, is independently hydrogen, C₁₋₄alkyl, or C₃₋₆cycloalkyl;
n is 1-3; and

E¹ and E² are each independently

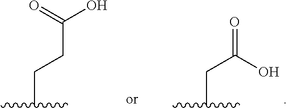 [SMILES: *CCC(=O)O ; *CC(=O)O]

In another aspect, A¹ is C₉₋₁₅alkyl. In another aspect, R¹, R², R³, and R⁴, at each occurrence, are each independently methyl or isopropyl. In another aspect, the peptide of formula (I) is a peptide of formula (I-a):

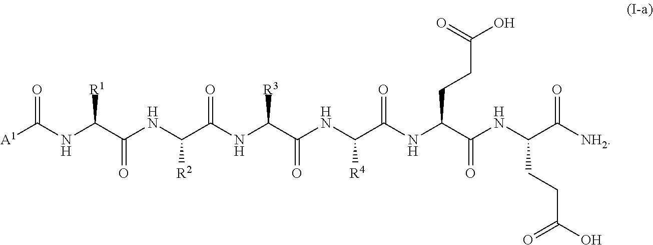

In another aspect, the peptide of formula (I) is:

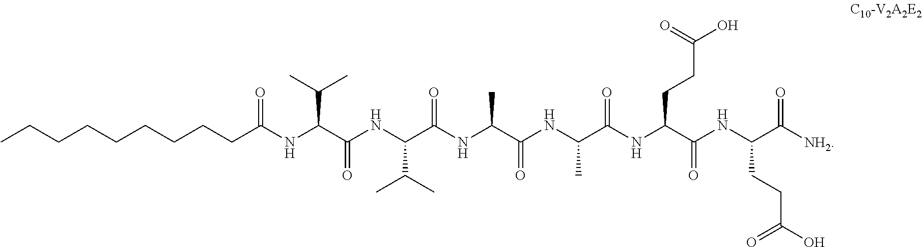 [SMILES: CCCCCCCCCC(=O)N[C@H](C(=O)N[C@H](C(=O)N[C@@H](C)C(=O)N[C@@H](C)C(=O)N[C@@H](CCC(=O)O)C(=O)N[C@@H](CCC(=O)O)C(N)=O)C(C)C)C(C)C]

In another aspect, in a solution having a pH of about 5, the hydrogel is intact. In another aspect, in a solution having a pH of about 7, the hydrogel disassembles. In another aspect, the glucagon analogue comprises one or more of: dasiglucagon or a depsi-glucagon analogue.

Another embodiment described herein is a pharmaceutical composition comprising glucagon or a glucagon analogue encapsulated within a hydrogel.

Another embodiment described herein is a method of treating an insulin disorder, the method comprising administering a therapeutically effective amount of the pharmaceutical composition comprising glucagon or a glucagon analogue encapsulated within a hydrogel to a subject in need thereof.

Another embodiment described herein is a method of modulating glucose levels in a subject, the method comprising: administering a therapeutically effective amount of a pharmaceutical composition to a subject in need thereof, the pharmaceutical composition comprising: glucagon or a glucagon analogue encapsulated within a hydrogel, the hydrogel comprising a peptide of formula (I):

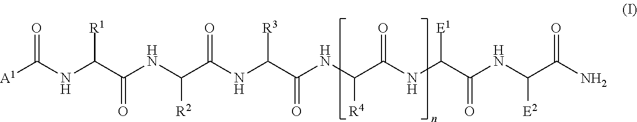

wherein:

A¹ is C₆₋₂₀alkyl;
R¹, R², R³, and R⁴, at each occurrence, are independently C₁_alkyl, C₃₋₆cycloalkyl, C₁₋₂haloalkyl, C₁₋₄hydroxyalkyl, halogen, —CN, —OR¹¹, —NHR¹¹, —CO₂R¹¹, —N(R¹¹)₂, —C(O)NHR¹¹, or —C(O)N(R¹¹)₂;
R¹¹, at each occurrence, is independently hydrogen, C₁₋₄alkyl, or C₃₋₆cycloalkyl;
n is 1-3; and
E¹ and E² are each independently

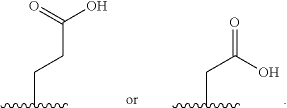 [SMILES: *CCC(=O)O ; *CC(=O)O]

In another aspect, the peptide of formula (I) is:

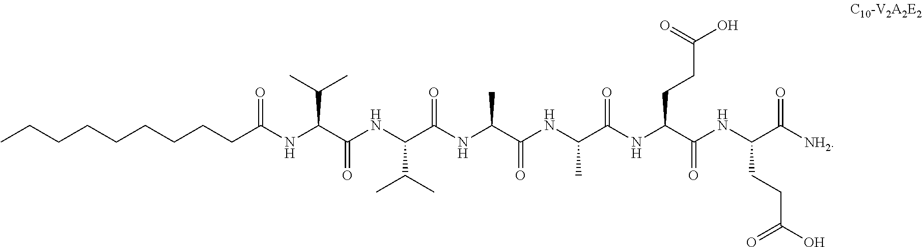 [SMILES: CCCCCCCCCC(=O)N[C@H](C(=O)N[C@H](C(=O)N[C@@H](C)C(=O)N[C@@H](C)C(=O)N[C@@H](CCC(=O)O)C(=O)N[C@@H](CCC(=O)O)C(N)=O)C(C)C)C(C)C]

In another aspect, the subject in need thereof is experiencing a hypoglycemic event. In another aspect, the subject in need thereof is at risk of experiencing a hypoglycemic event. In another aspect, the subject in need thereof has diabetes. In another aspect, the subject is normoglycemic or hyperglycemic, the glucagon or glucagon analogue is not released from the hydrogel. In another aspect, the subject is hypoglycemic, the glucagon or glucagon analogue is released from the hydrogel. In another aspect, the glucagon analogue comprises one or more of: dasiglucagon or a depsi-glucagon analogue. In another aspect, the therapeutically effective amount comprises 0.1-10 mg.

### DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 schematically shows embodiments described herein. Enzymatic control of molecular self-assembly and hydrogelation enables encapsulation and glucose-responsive delivery of a therapeutic to address low glucose.

FIG. 2A schematically illustrates the cyclic process of using glucose oxidase (GOₓ) as an actuator to convert glucose fuel to a pH stimulus directing and stabilizing peptide assemblies. In conditions of limited glucose, natural physiological buffering restores the material to its destabilized state. FIG. 2B shows a schematic illustrating that materials stabilized in the presence of a glucose fuel reverses the traditional paradigm in glucose-responsive materials, instead targeting material stability in states of normal glucose and dissolution in low glucose to release a glucagon therapeutic. FIG. 2C shows the structure of the C₁₀-V₂A₂E₂ peptide amphiphile (PA) that achieves self-assembly and hydrogelation under direction of a glucose fuel.

FIG. 3A-E show pH-dependent release of encapsulated 3 kDa FITC-dextran to screen different PA sequences. FIG. 3A shows pH-dependent release for C₁-V₂A₂E₂. FIG. 3B shows pH-dependent release for C₁₆-VA₃E₂. FIG. 3C shows pH-dependent release for C₁₆-A₂V₂E₂. FIG. 3D shows pH-dependent release for C₁₀-V₂A₂E₂. FIG. 3E shows pH-dependent release for C₁₀-VA₃E₂. For each sequence, A Release % (pH 7-5 at 5 hr) data is shown in Table 2.

FIG. 4A shows transmission electron microscopy images for assessing pH-dependent nanostructure formation of C₁₀—V₂A₂E₂ PA in films cast from 0.1% w/v PA solutions at various pH values. FIG. 4B shows near-UV circular dichroism spectra of C₁₀-V₂A₂E₂ PA at various pH values at a sub-gelation concentration of 0.1% w/v. FIG. 4C shows background-subtracted thioflavin-T (ThT) fluorescence, comparing C₁₀-V₂A₂E₂ PA (0.1% w/v) when changed rapidly from pH 5 to pH 7 to a sample maintained at pH 7 throughout. FIG. 4D shows pH-directed hydrogel formation at 1% w/v C₁₀-V₂A₂E₂ PA in physiologic buffers of assorted pH. Images collected 15 min following sample preparation. FIG. 4E shows plateau modulus for C₁₀-V₂A₂E₂ PA samples prepared at 1% w/v in a buffer of various pH (1% strain, 10 rad/s, average of 2 gels/group). FIG. 4F shows step-strain rheological testing of C₁₀-V₂A₂E₂ PA hydrogels prepared at pH 5 and cycled at a frequency of 10 rad/s between 1% and 100% strain.

FIG. 5A-B show Fourier transform infrared spectrometry (FTIR) analysis of samples at various pH monitoring signals attributed to β-sheet (˜1621 cm⁻¹) and random coil (˜1650 cm⁻¹). FIG. 5A shows normalized FTIR spectra that are offset on the y-axis to visualize changes with pH. FIG. 5B is a bar graph showing the ratio of the 1621 cm⁻¹ and 1650 cm⁻¹ peaks at different pH values.

FIG. 6A-B show strain sweep (FIG. 6A; 10 rad/s) and frequency sweep (FIG. 6B; 1% strain) graphs for 1 wt % C₁₀-V₂A₂E₂ hydrogel in pH 5 buffer.

FIG. 7A-B show strain sweep, frequency sweep, and time sweep (1% strain, 10 rad/s) graphs for 1 wt % C₁₀-V₂A₂E₂ hydrogel in all pH conditions (FIG. 7A) and glucose conditions (FIG. 7B). Average G′ values from the time sweep (bottom) are plotted for the bar graph in the main text.

FIG. 8A-B show rheological analysis/characterization (FIG. 8A) and circular dichroism spectra (FIG. 8B) performed for pH 7.4 buffer the presence of physiological calcium (0.9 mM) and magnesium (0.5 mM) compared to the same samples in a buffer of pH 7.4 and a buffer of pH 5 without addition of the divalent ions.

FIG. 9A-D shows scanning electron microscopy (SEM) images to visualize the highly porous architecture of the nanofibrillar hydrogel networks. Imaging was performed on a sample prepared by ethanol dehydration and critical point drying (FIG. 9A), with increasing magnification of specific regions shown in FIG. 9B-D.

FIG. 10A shows a glucose-directed hydrogel formation at 1% w/v C₁₀-V₂A₂E₂ PA with GOₓ in physiologic buffers at pH 7.4 and with assorted glucose concentrations. Images collected 24 h following sample preparation. FIG. 10B is a bar graph showing plateau modulus for C₁₀-V₂A₂E₂ PA samples prepared at 1% w/v in pH 7.4 buffer of various glucose concentrations (1% strain, 10 rad/s). FIG. 10C is a bar graph showing pH recorded by submersion of an electrode into hydrogel samples 24 h after under conditions of various glucose input.

FIG. 11A graphically shows glucose-dependent release of methoxycoumarin (MCA)-dasiglucagon from within 100 μL of 1% w/v C₁₀-V₂A₂E₂ PA hydrogels with GOₓ prepared initially in pH 5 buffer and then incubated in a bulk solution of 4 mL pH 7.5 buffer containing different levels of dissolved glucose. Data were fit to a standard first-order release model. FIG. 11B is a bar graph showing the total MCA-dasiglucagon released at 24 h, combined with MCA-dasiglucagon remaining within residual material after treatment of the system with concentrated base (gray bars in all cases). FIG. 11C is a bar graph showing the final pH of the release system after 24 h. FIG. 11D graphically shows step-change release over a period of four hours, beginning with hydrogels prepared at 100 mg/dL glucose and then exchanging the full 4 mL release buffer with a buffer containing 0 mg/dL glucose at 2 h (red point).

FIG. 12A graphically shows dasiglucagon release over a period of 7 hours from PA hydrogels prepared with and without GOₓ and incubated in a bulk buffer containing 100 mg/dL (n=3/group). FIG. 12B shows the gels without GOₓ were completely dissolved by 9 h, whereas the gels with GOₓ became more transparent but otherwise did not show significant reduction in size.

FIG. 13 graphically shows the initial drop in the bulk pH when 100 μL PA hydrogels were incubated in 4 mL of a pH 7.4 buffer containing different concentrations (mg/dL) of glucose (n=3/group) over a period of 10 hours.

FIG. 14 graphically shows GOₓ activity assessment via repeated pH change over a period of 10 days when 100 μL PA hydrogels were incubated in 4 mL of a pH 7.4 salt solution recharged daily with a fresh bulk phase containing 200 mg/dL glucose. pH was sampled immediately before and after bulk solution exchange. GOₓ in the hydrogel reduces pH repeatedly for at least 10 days.

FIG. 15 shows circular dichroism spectra of dasiglucagon incubated for 7 days in pH 5 buffer and monitored for preservation of active structure, confirming no formation of degradation or amyloid products for at least 1 week under these conditions.

FIG. 16 shows release studies with full buffer exchange. All samples were incubated at 100 mg/dL for 2 h, at which time half had buffer fully exchanged for another 100 mg/dL while half had buffer exchanged for 0 mg/dL.

FIG. 17A shows a cartoon schematic overview with data for the full in vivo experimental model to assess prophylactic hypoglycemia correction with C₁₀-V₂A₂E₂ PA hydrogels. Streptozotocin (STZ) diabetic mice were fasted and then administered insulin detemir to stabilize blood glucose within a normal range. After 4 h, treatments were then administered (t=0) and blood glucose was monitored. At 2 h after treating with buffer, dasiglucagon, or PA hydrogel, an insulin overdose was performed. Blood glucose was monitored throughout the study. A dashed line is drawn at 60 mg/dL for visualization of the approximate region characterized clinically as mild hypoglycemia (<70 mg/dL but >54 mg/dL). The extent and duration of hypoglycemia was evaluated between treatments with arrows noting the timing of observed deaths. FIG. 17B is a bar graph showing the nadir (lowest) blood glucose reading for the three different treatment groups. FIG. 17C shows the final blood glucose measured at 300 minutes for the three different treatment groups. Each treatment group was n=9 mice, error bars indicate SEM for each group, and statistical analysis was performed using ANOVA with multiple comparisons post-hoc testing.

FIG. 18A-C show plotted Blood Glucose Level (BGL) results of the hypoglycemic region over a time period of 120-300 minutes for each individual mouse in the study (n=9/group). FIG. 18A shows the plotted results with buffer; FIG. 18B shows the plotted results with dasiglucagon; FIG. 18C shows the plotted results with PA gel and dasiglucagon.

FIG. 19A-B show experimental results assessing the role of GOₓ actuation in function of the hydrogel system with glucagon delivered in a PA-gel prepared at pH 5 with (n=5) and without (n=6) GOₓ. These studies were conducted with a modification to the typical control strategy used for other studies herein. FIG. 19A is a scatterplot graph showing initial glucose control was achieved with 0.75 IU/kg insulin detemir, and two hours following gel administration insulin (2 IU/kg) was administered to induce hypoglycemia. FIG. 19B is a bar graph showing the comparison of the extent of hypoglycemia (nadir) between the two groups (hydrogel with and without GOₓ) (**P<0.01 by t-test).

FIG. 20 shows the circular dichroism spectrum of dasiglucagon at 0.05 mg/mL in 50 mM phosphate buffer (pH 7).

FIG. 21A-B show fluorescent properties of MCA-dasiglucagon. FIG. 21A shows normalized spectra of absorbance (excitation) and fluorescence (emission). FIG. 21B shows a standard curve used to determine concentration (μg/mL).

### DETAILED DESCRIPTION

Unless otherwise defined, all technical and scientific terms used herein have the same meaning as commonly understood by one of ordinary skill in the art. For example, any nomenclatures used in connection with, and techniques of chemistry, biochemistry, molecular biology, immunology, microbiology, genetics, cell and tissue culture, and protein and nucleic acid chemistry described herein are well known and commonly used in the art. In case of conflict, the present disclosure, including definitions, will control. Exemplary methods and materials are described below, although methods and materials similar or equivalent to those described herein can be used in practice or testing of the embodiments and aspects described herein.

As used herein, the terms “amino acid,” “nucleotide,” “polynucleotide,” “vector,” “polypeptide,” and “protein” have their common meanings as would be understood by a biochemist of ordinary skill in the art. Standard single letter nucleotides (A, C, G, T, U) and standard single letter amino acids (A, C, D, E, F, G, H, I, K, L, M, N, P, Q, R, S, T, V, W, or Y) are used herein.

As used herein, the terms such as “include,” “including,” “contain,” “containing,” “having,” and the like mean “comprising.” The present disclosure also contemplates other embodiments “comprising,” “consisting essentially of,” and “consisting of” the embodiments or elements presented herein, whether explicitly set forth or not.

As used herein, the term “a,” “an,” “the” and similar terms used in the context of the disclosure (especially in the context of the claims) are to be construed to cover both the singular and plural unless otherwise indicated herein or clearly contradicted by the context. In addition, “a,” “an,” or “the” means “one or more” unless otherwise specified.

As used herein, the term “or” can be conjunctive or disjunctive.

As used herein, the term “and/or” refers to both the conjunctive and disjunctive.

As used herein, the term “substantially” means to a great or significant extent, but not completely.

As used herein, the term “about” or “approximately” as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In one aspect, the term “about” refers to any values, including both integers and fractional components that are within a variation of up to ±10% of the value modified by the term “about.” Alternatively, “about” can mean within 3 or more standard deviations, per the practice in the art. Alternatively, such as with respect to biological systems or processes, the term “about” can mean within an order of magnitude, in some embodiments within 5-fold, and in some embodiments within 2-fold, of a value. As used herein, the symbol “˜” means “about” or “approximately.”

All ranges disclosed herein include both end points as discrete values as well as all integers and fractions specified within the range. For example, a range of 0.1-2.0 includes 0.1, 0.2, 0.3, 0.4 . . . 2.0. If the end points are modified by the term “about,” the range specified is expanded by a variation of up to ±10% of any value within the range or within 3 or more standard deviations, including the end points.

As used herein, the terms “active ingredient” or “active pharmaceutical ingredient” refer to a pharmaceutical agent, active ingredient, compound, or substance, compositions, or mixtures thereof, that provide a pharmacological, often beneficial, effect.

As used herein, the terms “control,” or “reference” are used herein interchangeably. A “reference” or “control” level may be a predetermined value or range, which is employed as a baseline or benchmark against which to assess a measured result. “Control” also refers to control experiments or control cells.

As used herein, the term “dose” denotes any form of an active ingredient formulation or composition, including cells, that contains an amount sufficient to initiate or produce a therapeutic effect with at least one or more administrations. “Formulation” and “composition” are used interchangeably herein.

As used herein, the term “prophylaxis” refers to preventing or reducing the progression of a disorder, either to a statistically significant degree or to a degree detectable by a person of ordinary skill in the art.

As used herein, the terms “effective amount” or “therapeutically effective amount,” refers to a substantially non-toxic, but sufficient amount of an action, agent, composition, or cell(s) being administered to a subject that will prevent, treat, or ameliorate to some extent one or more of the symptoms of the disease or condition being experienced or that the subject is susceptible to contracting. The result can be the reduction or alleviation of the signs, symptoms, or causes of a disease, or any other desired alteration of a biological system. An effective amount may be based on factors individual to each subject, including, but not limited to, the subject's age, size, type or extent of disease, stage of the disease, route of administration, the type or extent of supplemental therapy used, ongoing disease process, and type of treatment desired.

As used herein, the term “subject” refers to an animal. Typically, the subject is a mammal. A subject also refers to primates (e.g., humans, male or female; infant, adolescent, or adult), non-human primates, rats, mice, rabbits, pigs, cows, sheep, goats, horses, dogs, cats, fish, birds, and the like. In one embodiment, the subject is a primate. In one embodiment, the subject is a human.

As used herein, a subject is “in need of treatment” if such subject would benefit biologically, medically, or in quality of life from such treatment. A subject in need of treatment does not necessarily present symptoms, particular in the case of preventative or prophylaxis treatments.

As used herein, the terms “inhibit,” “inhibition,” or “inhibiting” refer to the reduction or suppression of a given biological process, condition, symptom, disorder, or disease, or a significant decrease in the baseline activity of a biological activity or process.

As used herein, “treatment” or “treating” refers to prophylaxis of, preventing, suppressing, repressing, reversing, alleviating, ameliorating, or inhibiting the progress of biological process including a disorder or disease, or completely eliminating a disease. A treatment may be either performed in an acute or chronic way. The term “treatment” also refers to reducing the severity of a disease or symptoms associated with such disease prior to affliction with the disease. “Repressing” or “ameliorating” a disease, disorder, or the symptoms thereof involves administering a cell, composition, or compound described herein to a subject after clinical appearance of such disease, disorder, or its symptoms. “Prophylaxis of” or “preventing” a disease, disorder, or the symptoms thereof involves administering a cell, composition, or compound described herein to a subject prior to onset of the disease, disorder, or the symptoms thereof. “Suppressing” a disease or disorder involves administering a cell, composition, or compound described herein to a subject after induction of the disease or disorder thereof but before its clinical appearance or symptoms thereof have manifest.

As used herein, the term “peptide amphiphile” or “PA” refers to a peptide-based molecule that is capable of self-assembling into supramolecular nanostructures including, but not limited to, spherical micelles, twisted ribbons, and high-aspect-ratio nanofibers.

As used herein, the term “amphiphilic” refers to a molecule or species having both hydrophobic and hydrophilic character.

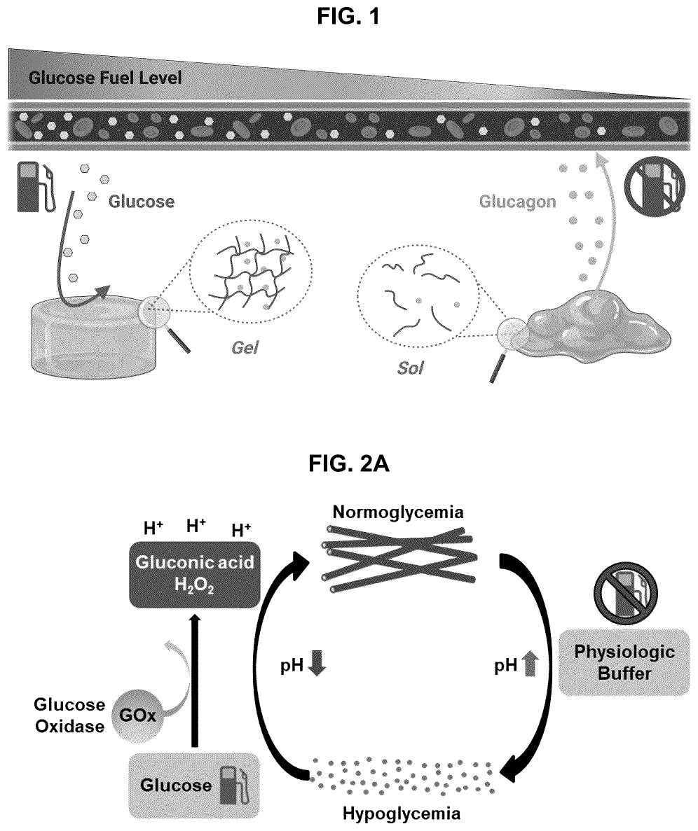

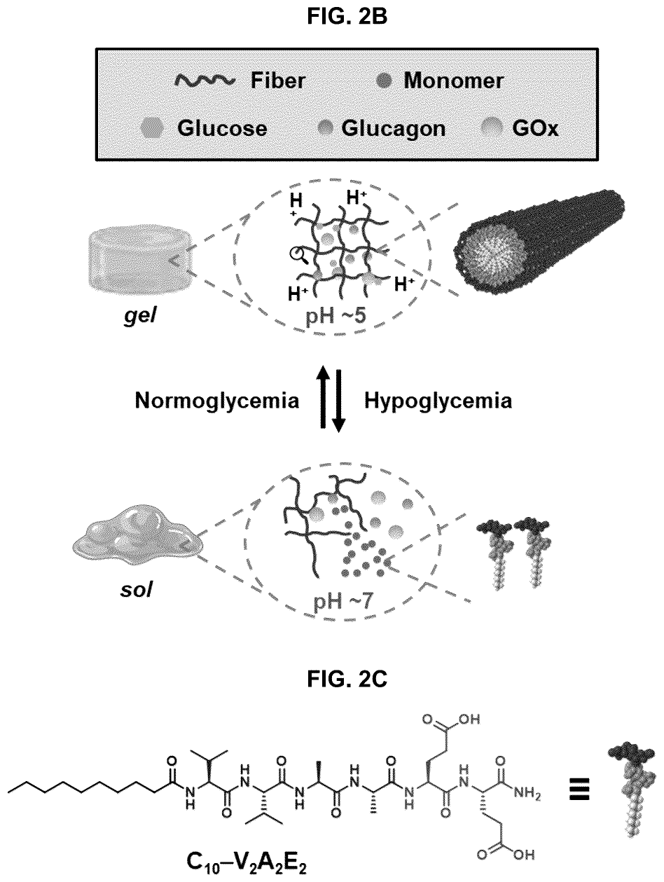

Described herein is a nanofibrillar assembly that leverages GOₓ to drive non-equilibrium network formation of a peptide hydrogelator through the localized reduction in pH achieved by consumption of physiologic glucose “fuel” (FIG. 1-2). In the absence of sufficient glucose fuel, as upon onset of hypoglycemia, a neutral-buffered physiological milieu acts as a directive to promote gel dissolution through molecular disassembly, restoring the equilibrium state. This approach contrasts with the preponderance of literature in glucose-responsive materials design that seeks to use glucose to drive material disassembly or erosion for the release of insulin, here instead offering a route to transiently stabilize nanofibrillar hydrogels in the presence of glucose. Accordingly, the present strategy is explored using glucose-stabilized materials for glucose-responsive delivery of glucagon as a preventative route to combat the subsequent onset of hypoglycemia.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All of the various embodiments, aspects, and options disclosed herein can be combined in any variations or iterations. The scope of the compositions, formulations, methods, and processes described herein include all actual or potential combinations of embodiments, aspects, options, examples, and preferences herein described. The exemplary compositions and formulations described herein may omit any component, substitute any component disclosed herein, or include any component disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. All patents and publications cited herein are incorporated by reference herein for the specific teachings thereof.

### Compounds

In one aspect, the invention provides hydrogels. Exemplary hydrogels of the present invention comprise a peptide (e.g., a peptide of formula (I)) and glucagon or a glucagon analogue. In various instances, the glucagon or analogue thereof is encapsulated within the hydrogel. In various instances, in a solution having a pH of about 5, the hydrogel is intact. In various instances, in a solution having a pH of about 7, the hydrogel disassembles.

### Peptides

In one aspect, the invention provides peptides of formula (I):

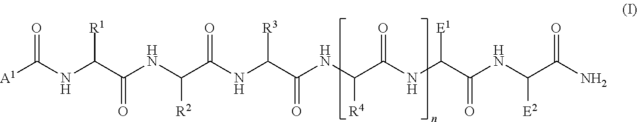

wherein:
A¹, R¹, R², R³, R⁴, R¹¹, n, E¹ and E² are as defined herein.

In various instances, A¹ is C₆₋₂₀alkyl, wherein:
R¹, R², R³, and R⁴, at each occurrence, are independently C₁₋₆alkyl, C₃₋₆cycloalkyl, C₁₋₂haloalkyl, C₁₋₄hydroxyalkyl, halogen, —CN, —OR¹¹, —NHR¹¹, —CO₂R¹¹, —N(R¹¹)₂, —C(O)NHR¹¹, or —C(O)N(R¹¹)₂;
R¹¹, at each occurrence, is independently hydrogen, C₁₋₄alkyl, or C₃₋₆cycloalkyl;
n is 1-3; and
E¹ and E² are each independently

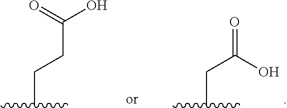 [SMILES: *CCC(=O)O ; *CC(=O)O]

In various instances, A¹ is C₉₋₁₅alkyl.

In various instances, A¹ is linear.

In various instances, R¹, R², R³, and R⁴, at each occurrence, are each independently C₁₋₄alkyl.

In various instances, R¹, R², R³, and R⁴, at each occurrence, are each independently methyl or isopropyl.

In various instances,

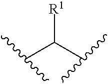

is

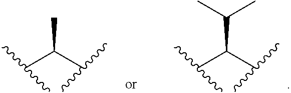

In various instances,

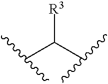

is

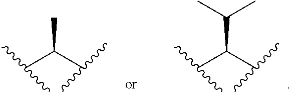

In various instances,

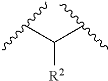

is

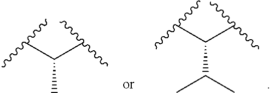

In various instances,

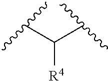

is

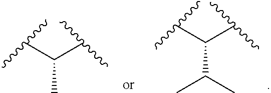

In various instances, E¹ and E² are each

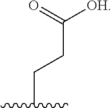 [SMILES: *CCC(=O)O]

In various instances,

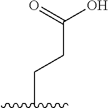 [SMILES: *CCC(=O)O]

is

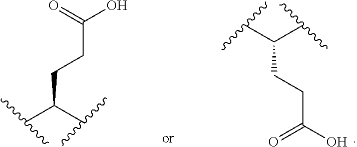

In various instances, the peptide of formula (I) is a peptide of formula (I-a)

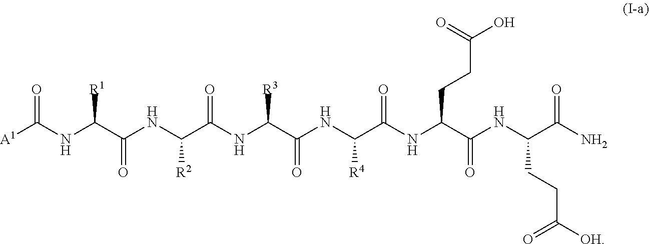

In various instances, the peptide of formula (I) is a peptide selected from the group consisting of:

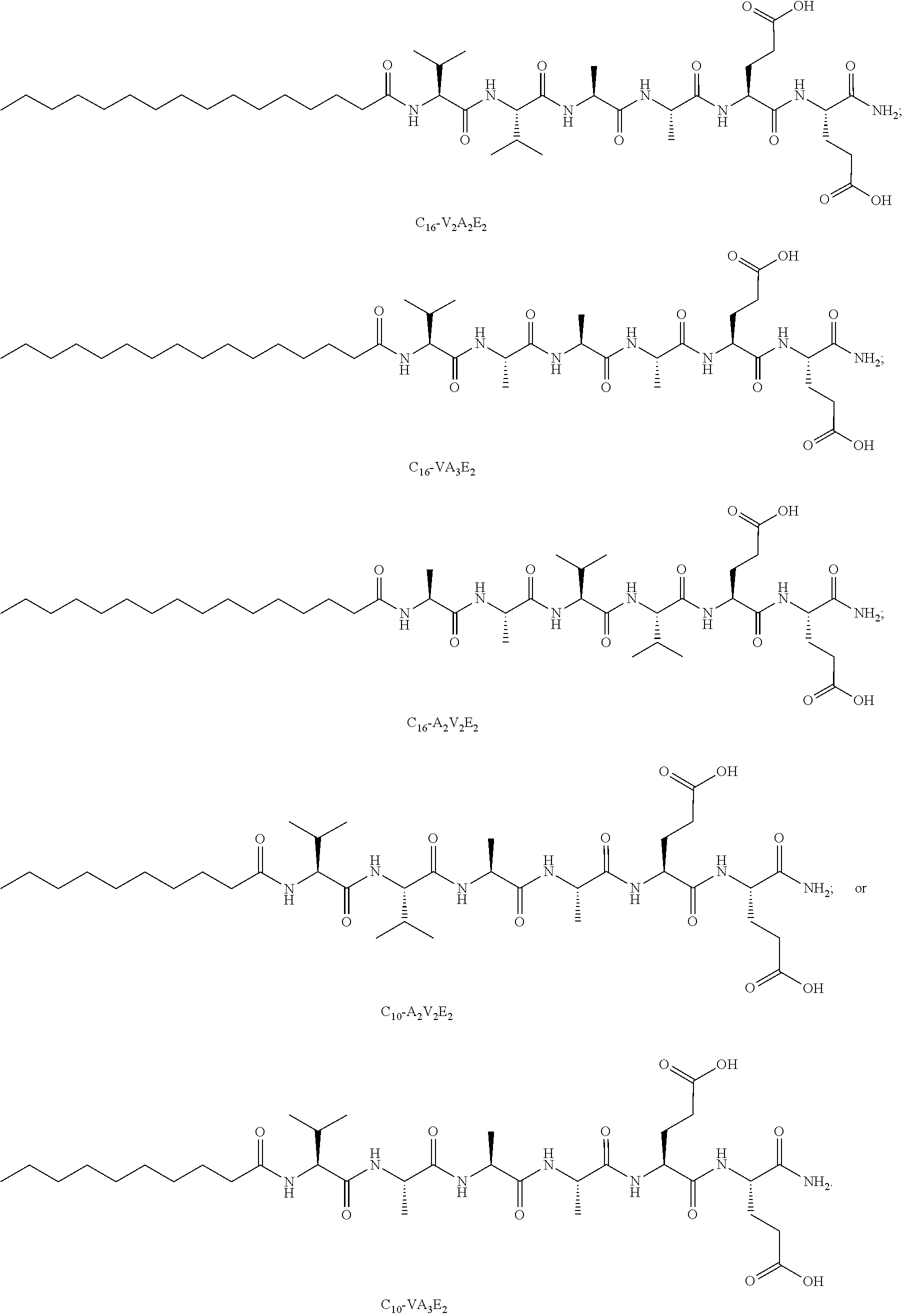

Compound names can be assigned by using Struct=Name naming algorithm as part of CHEMDRAW® ULTRA.

The compound may exist as a stereoisomer wherein asymmetric or chiral centers are present. The stereoisomer is “R” or “S” depending on the configuration of substituents around the chiral carbon atom. The terms “R” and “S” used herein are configurations as defined in IUPAC 1974 Recommendations for Section E, Fundamental Stereochemistry, in Pure Appl. Chem., 1976, 45: 13-30. The disclosure contemplates various stereoisomers and mixtures thereof and these are specifically included within the scope of this invention. Stereoisomers include enantiomers and diastereomers, and mixtures of enantiomers or diastereomers. Individual stereoisomers of the compounds may be prepared synthetically from commercially available starting materials, which contain asymmetric or chiral centers or by preparation of racemic mixtures followed by methods of resolution well-known to those of ordinary skill in the art. These methods of resolution are exemplified by (1) attachment of a mixture of enantiomers to a chiral auxiliary, separation of the resulting mixture of diastereomers by recrystallization or chromatography and optional liberation of the optically pure product from the auxiliary as described in Furniss, Hannaford, Smith, and Tatchell, “Vogel's Textbook of Practical Organic Chemistry”, 5th edition (1989), Longman Scientific & Technical, Essex CM20 2JE, England, or (2) direct separation of the mixture of optical enantiomers on chiral chromatographic columns or (3) fractional recrystallization methods.

It should be understood that the compound may possess tautomeric forms, as well as geometric isomers, and that these also constitute an aspect of the invention.

In the compounds of formula (I), formula (II), and any subformulas, any “hydrogen” or “H,” whether explicitly recited or implicit in the structure, encompasses hydrogen isotopes ¹H (protium) and ²H (deuterium).

The present disclosure also includes isotopically-labeled compounds (e.g., deuterium labeled), where an atom in the isotopically-labeled compound is specified as a particular isotope of the atom. Examples of isotopes suitable for inclusion in the compounds of the invention are hydrogen, carbon, nitrogen, oxygen, phosphorus, sulfur, fluorine, and chlorine, such as, but not limited to ²H, ³H, ¹³C, ¹⁴C, ¹⁵N, ¹⁸O, ¹⁷O, ³¹P, ³²P, ³⁵S, ¹⁸F, and ³⁶Cl, respectively.

Isotopically-enriched forms of compounds of formula (I), or any subformulas, may generally be prepared by conventional techniques known to those skilled in the art or by processes analogous to those described in the accompanying Examples using an appropriate isotopically-enriched reagent in place of a non-isotopically-enriched reagent. The extent of isotopic enrichment can be characterized as a percent incorporation of a particular isotope at an isotopically-labeled atom (e.g., % deuterium incorporation at a deuterium label).

### Glucagon and Glucagon Analogues

Glucagon administered at low doses may prevent insulin-induced hypoglycemia or improve the ability to recover from hypoglycemia. However, glucagon is of limited use in pharmaceuticals due to fast clearance from circulation with a half-life of approximately 5 min. Compared to glucagon, glucagon-like analogues (i.e., “glucagon analogues”) may demonstrate improved physical stability toward gel and fibril formation, improved chemical stability and increased half-life, while also showing improved aqueous solubility at neutral pH or slightly basic pH. Glucagon analogues mimic the endogenous hormone glucagon-like peptide 1 (GLP-1), a gastrointestinal hormone that is released into the circulation in response to ingested nutrients. Various exemplary glucagon-based analogues are described in U.S. Pat. No. 9,486,506 B2. In various instances, the glucagon analogue comprises one or more of: dasiglucagon or a depsi-glucagon analogue. Various exemplary depsi-glucagon analogues are described in international patent publication WO 2017/210168 A1.

### Pharmaceutical Salts

The disclosed compounds may exist as pharmaceutically acceptable salts. The term “pharmaceutically acceptable salt” refers to salts or zwitterions of the compounds which are water or oil-soluble or dispersible, suitable for treatment of disorders without undue toxicity, irritation, and allergic response, commensurate with a reasonable benefit/risk ratio and effective for their intended use. The salts may be prepared during the final isolation and purification of the compounds or separately by reacting an amino group of the compounds with a suitable acid. For example, a compound may be dissolved in a suitable solvent, such as but not limited to methanol and water and treated with at least one equivalent of an acid, like hydrochloric acid. The resulting salt may precipitate out and be isolated by filtration and dried under reduced pressure. Alternatively, the solvent and excess acid may be removed under reduced pressure to provide a salt. Representative salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, isethionate, fumarate, lactate, maleate, methanesulfonate, naphthylenesulfonate, nicotinate, oxalate, pamoate, pectinate, persulfate, 3-phenylpropionate, picrate, oxalate, maleate, pivalate, propionate, succinate, tartrate, thrichloroacetate, trifluoroacetate, glutamate, para-toluenesulfonate, undecanoate, hydrochloric, hydrobromic, sulfuric, phosphoric and the like. The amino groups of the compounds may also be quaternized with alkyl chlorides, bromides, and iodides such as methyl, ethyl, propyl, isopropyl, butyl, lauryl, myristyl, stearyl and the like.

Basic addition salts may be prepared during the final isolation and purification of the disclosed compounds by reaction of a carboxyl group with a suitable base such as the hydroxide, carbonate, or bicarbonate of a metal cation such as lithium, sodium, potassium, calcium, magnesium, or aluminum, or an organic primary, secondary, or tertiary amine. Quaternary amine salts can be prepared, such as those derived from methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine and N,N′-dibenzylethylenediamine, ethylenediamine, ethanolamine, diethanolamine, piperidine, piperazine, and the like.

### General Synthesis of Compounds

Optimum reaction conditions and reaction times for each individual step can vary depending on the particular reactants employed and substituents present in the reactants used. Specific procedures are provided in the Examples section. Reactions can be worked up in the conventional manner, e.g., by eliminating the solvent from the residue and further purified according to methodologies generally known in the art such as, but not limited to, crystallization, distillation, extraction, trituration, and chromatography. Unless otherwise described, the starting materials and reagents are either commercially available or can be prepared by one skilled in the art from commercially available materials using methods described in the chemical literature.

Starting materials, if not commercially available, can be prepared by procedures selected from standard organic chemical techniques, techniques that are analogous to the synthesis of known, structurally similar compounds, or techniques that are analogous to the above-described schemes or the procedures described in the synthetic examples section.

Routine experimentations, including appropriate manipulation of the reaction conditions, reagents and sequence of the synthetic route, protection of any chemical functionality that cannot be compatible with the reaction conditions, and deprotection at a suitable point in the reaction sequence of the method are included in the scope of the invention. Suitable protecting groups and the methods for protecting and deprotecting different substituents using such suitable protecting groups are well known to those skilled in the art; examples of which can be found in PGM Wuts and TW Greene, in Greene's book titled Protective Groups in Organic Synthesis (4^{th} ed.), John Wiley & Sons, NY (2006), which is incorporated herein by reference in its entirety. Synthesis of the compounds of the invention can be accomplished by methods analogous to those described in the synthetic schemes described hereinabove and in specific examples.

When an optically active form of a disclosed compound is required, it can be obtained by carrying out one of the procedures described herein using an optically active starting material (prepared, for example, by asymmetric induction of a suitable reaction step), or by resolution of a mixture of the stereoisomers of the compound or intermediates using a standard procedure (such as chromatographic separation, recrystallization, or enzymatic resolution).

Similarly, when a pure geometric isomer of a compound is required, it can be obtained by carrying out one of the above procedures using a pure geometric isomer as a starting material, or by resolution of a mixture of the geometric isomers of the compound or intermediates using a standard procedure such as chromatographic separation.

It can be appreciated that the synthetic schemes and specific examples as described are illustrative and are not to be read as limiting the scope of the invention as it is defined in the appended claims. All alternatives, modifications, and equivalents of the synthetic methods and specific examples are included within the scope of the claims.

### Pharmaceutical Compositions

Pharmaceutical compositions of the present invention comprise glucagon or a glucagon analogue encapsulated within the hydrogels disclosed herein (i.e., “hydrogel-encapsulated glucagon/glucagon analogue”).

The hydrogel-encapsulated glucagon/glucagon analogue may be incorporated into pharmaceutical compositions suitable for administration to a subject (such as a patient, which may be a human or non-human). The pharmaceutical compositions may include a “therapeutically effective amount” or a “prophylactically effective amount” of the active agent (glucagon or glucagon analogue). A “therapeutically effective amount” refers to an amount effective, at dosages and for periods of time necessary, to achieve the desired therapeutic result. A therapeutically effective amount of the composition may be determined by a person skilled in the art and may vary according to factors such as the disease state, age, sex, and weight of the individual, and the ability of the composition to elicit a desired response in the individual. A “therapeutically effective amount” is also one in which any toxic or detrimental effects are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

The pharmaceutical compositions may include pharmaceutically acceptable carriers. The term “pharmaceutically acceptable carrier,” as used herein, means a non-toxic, inert solid, semisolid or liquid filler, diluent, encapsulating material, or formulation auxiliary of any type. Some examples of materials which can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; glycols; such as propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Thus, the hydrogels and their physiologically acceptable salts and solvates may be formulated for administration by, for example, solid dosing, eyedrop, in a topical oil-based formulation, injection, inhalation (either through the mouth or the nose), implants, or oral, buccal, parenteral, or rectal administration. Techniques and formulations may generally be found in “Remington's Pharmaceutical Sciences,” (Meade Publishing Co., Easton, Pa.). Therapeutic compositions must typically be sterile and stable under the conditions of manufacture and storage.

The route by which the hydrogel-encapsulated glucagon/glucagon analogue is administered, and the form of the composition will dictate the type of carrier to be used. The composition may be in a variety of forms, suitable, for example, for systemic administration (e.g., oral, rectal, nasal, sublingual, buccal, implants, or parenteral) or topical administration (e.g., dermal, pulmonary, nasal, aural, ocular, liposome delivery systems, or iontophoresis).

Carriers for systemic administration typically include at least one of diluents, lubricants, binders, disintegrants, colorants, flavors, sweeteners, antioxidants, preservatives, glidants, solvents, suspending agents, wetting agents, surfactants, combinations thereof, and others. All carriers are optional in the compositions. Suitable diluents include sugars such as glucose, lactose, dextrose, and sucrose; diols such as propylene glycol; calcium carbonate; sodium carbonate; sugar alcohols, such as glycerin; mannitol; and sorbitol. The amount of diluent(s) in a systemic or topical composition is typically about 50 to about 90%.

Suitable lubricants include silica, talc, stearic acid and its magnesium salts and calcium salts, calcium sulfate; and liquid lubricants such as polyethylene glycol and vegetable oils such as peanut oil, cottonseed oil, sesame oil, olive oil, corn oil and oil of theobroma. The amount of lubricant(s) in a systemic or topical composition is typically about 5 to about 10%.

Suitable binders include polyvinyl pyrrolidone; magnesium aluminum silicate; starches such as corn starch and potato starch; gelatin; tragacanth; and cellulose and its derivatives, such as sodium carboxymethylcellulose, ethyl cellulose, methylcellulose, microcrystalline cellulose, and sodium carboxymethylcellulose. The amount of binder(s) in a systemic composition is typically about 5 to about 50%.

Suitable disintegrants include agar, alginic acid and the sodium salt thereof, effervescent mixtures, croscarmellose, crospovidone, sodium carboxymethyl starch, sodium starch glycolate, clays, and ion exchange resins. The amount of disintegrant(s) in a systemic or topical composition is typically about 0.1 to about 10%. Suitable colorants include a colorant such as an FD&C dye. When used, the amount of colorant in a systemic or topical composition is typically about 0.005 to about 0.1%. Suitable flavors include menthol, peppermint, and fruit flavors. The amount of flavor(s), when used, in a systemic or topical composition is typically about 0.1 to about 1.0%.

Suitable sweeteners include aspartame and saccharin. The amount of sweetener(s) in a systemic or topical composition is typically about 0.001 to about 1%. Suitable antioxidants include butylated hydroxyanisole (“BHA”), butylated hydroxytoluene (“BHT”), and vitamin E. The amount of antioxidant(s) in a systemic or topical composition is typically about 0.1 to about 5%. Suitable preservatives include benzalkonium chloride, methyl paraben and sodium benzoate. The amount of preservative(s) in a systemic or topical composition is typically about 0.01 to about 5%. Suitable glidants include silicon dioxide. The amount of glidant(s) in a systemic or topical composition is typically about 1 to about 5%.

Suitable solvents include water, isotonic saline, ethyl oleate, glycerine, hydroxylated castor oils, alcohols such as ethanol, and phosphate buffer solutions. The amount of solvent(s) in a systemic or topical composition is typically from about 0 to about 100%. Suitable suspending agents include AVICEL RC-591 (from FMC Corporation of Philadelphia, PA) and sodium alginate. The amount of suspending agent(s) in a systemic or topical composition is typically about 1 to about 8%. Suitable surfactants include lecithin, Polysorbate 80, and sodium lauryl sulfate, and the TWEENS from Atlas Powder Company of Wilmington, Delaware. Suitable surfactants include those disclosed in the C.T.F.A. Cosmetic Ingredient Handbook, 1992, pp. 587-592; Remington's Pharmaceutical Sciences, 15th Ed. 1975, pp. 335-337; and McCutcheon's Volume 1, Emulsifiers & Detergents, 1994, North American Edition, pp. 236-239. The amount of surfactant(s) in the systemic or topical composition is typically about 0.1% to about 5%.

Although the amounts of components in the systemic compositions may vary depending on the type of systemic composition prepared, in general, systemic compositions include 0.01% to 50% of actives and 50% to 99.99% of one or more carriers. Compositions for parenteral administration typically include 0.1% to 10% of actives and 90% to 99.9% of a carrier including a diluent and a solvent.

Compositions for oral administration can have various dosage forms. For example, solid forms include tablets, capsules, granules, and bulk powders. These oral dosage forms include a safe and effective amount, usually at least about 5%, and more particularly from about 25% to about 50% of actives. The oral dosage compositions include about 50% to about 95% of carriers, and more particularly, from about 50% to about 75%.

Tablets can be compressed, tablet triturates, enteric-coated, sugar-coated, film-coated, or multiple-compressed. Tablets typically include an active component, and a carrier comprising ingredients selected from diluents, lubricants, binders, disintegrants, colorants, flavors, sweeteners, glidants, and combinations thereof. Specific diluents include calcium carbonate, sodium carbonate, mannitol, lactose, and cellulose. Specific binders include starch, gelatin, and sucrose. Specific disintegrants include alginic acid and croscarmellose. Specific lubricants include magnesium stearate, stearic acid, and talc. Specific colorants are the FD&C dyes, which can be added for appearance. Chewable tablets preferably contain sweeteners such as aspartame and saccharin, or flavors such as menthol, peppermint, fruit flavors, or a combination thereof.

Capsules (including implants, time release and sustained release formulations) typically include an active and a carrier including one or more diluents disclosed above in a capsule comprising gelatin. Granules typically comprise an active, and preferably glidants such as silicon dioxide to improve flow characteristics. Implants can be of the biodegradable or the non-biodegradable type.

The selection of ingredients in the carrier for oral compositions depends on secondary considerations like taste, cost, and shelf stability, which are not critical for the purposes of this invention. Solid compositions may be coated by conventional methods, typically with pH or time-dependent coatings, such that the hydrogel-encapsulated glucagon/glucagon analogue is released in the gastrointestinal tract in the vicinity of the desired application, or at various points and times to extend the desired action. The coatings typically include one or more components selected from the group consisting of cellulose acetate phthalate, polyvinyl acetate phthalate, hydroxypropyl methyl cellulose phthalate, ethyl cellulose, EUDRAGIT coatings (available from Rohm & Haas G.M.B.H. of Darmstadt, Germany), waxes and shellac.

Compositions for oral administration can have liquid forms. For example, suitable liquid forms include aqueous solutions, emulsions, suspensions, solutions reconstituted from non-effervescent granules, suspensions reconstituted from non-effervescent granules, effervescent preparations reconstituted from effervescent granules, elixirs, tinctures, syrups, and the like. Liquid orally administered compositions typically include the hydrogel-encapsulated glucagon/glucagon analogue and a carrier, namely, a carrier selected from diluents, colorants, flavors, sweeteners, preservatives, solvents, suspending agents, and surfactants. Peroral liquid compositions preferably include one or more ingredients selected from colorants, flavors, and sweeteners.

Other compositions useful for attaining systemic delivery of the subject compounds include sublingual, buccal and nasal dosage forms. Such compositions typically include one or more of soluble filler substances such as diluents including sucrose, sorbitol, and mannitol; and binders such as acacia, microcrystalline cellulose, carboxymethyl cellulose, and hydroxypropyl methylcellulose. Such compositions may further include lubricants, colorants, flavors, sweeteners, antioxidants, and glidants.

The disclosed compositions can be topically administered. Topical compositions that can be applied locally to the skin may be in any form including solids, solutions, oils, creams, ointments, gels, lotions, shampoos, leave-on and rinse-out hair conditioners, milks, cleansers, moisturizers, sprays, skin patches, and the like. Topical compositions include: a disclosed hydrogel and a carrier. The carrier of the topical composition preferably aids penetration of the hydrogels into the skin. The carrier may further include one or more optional components.

The amount of the carrier employed in conjunction with the hydrogel-encapsulated glucagon/glucagon analogue is sufficient to provide a practical quantity of composition for administration per unit dose of the medicament. Techniques and compositions for making dosage forms useful in the methods of this invention are described in the following references: Modern Pharmaceutics, Chapters 9 and 10, Banker & Rhodes, eds. (1979); Lieberman et al., Pharmaceutical Dosage Forms: Tablets (1981); and Ansel, Introduction to Pharmaceutical Dosage Forms, 2^{nd} ed., (1976).

A carrier may include a single ingredient or a combination of two or more ingredients. In the topical compositions, the carrier includes a topical carrier. Suitable topical carriers include one or more ingredients selected from phosphate buffered saline, isotonic water, deionized water, monofunctional alcohols, symmetrical alcohols, aloe vera gel, allantoin, glycerin, vitamin A and E oils, mineral oil, propylene glycol, PPG-2 myristyl propionate, dimethyl isosorbide, castor oil, combinations thereof, and the like. More particularly, carriers for skin applications include propylene glycol, dimethyl isosorbide, and water, and even more particularly, phosphate buffered saline, isotonic water, deionized water, monofunctional alcohols, and symmetrical alcohols.

The carrier of a topical composition may further include one or more ingredients selected from emollients, propellants, solvents, humectants, thickeners, powders, fragrances, pigments, and preservatives, all of which are optional.

Suitable emollients include stearyl alcohol, glyceryl monoricinoleate, glyceryl monostearate, propane-1,2-diol, butane-1,3-diol, mink oil, cetyl alcohol, isopropyl isostearate, stearic acid, isobutyl palmitate, isocetyl stearate, oleyl alcohol, isopropyl laurate, hexyl laurate, decyl oleate, octadecan-2-ol, isocetyl alcohol, cetyl palmitate, di-n-butyl sebacate, isopropyl myristate, isopropyl palmitate, isopropyl stearate, butyl stearate, polyethylene glycol, triethylene glycol, lanolin, sesame oil, coconut oil, arachis oil, castor oil, acetylated lanolin alcohols, petroleum, mineral oil, butyl myristate, isostearic acid, palmitic acid, isopropyl linoleate, lauryl lactate, myristyl lactate, decyl oleate, myristyl myristate, and combinations thereof. Specific emollients for skin include stearyl alcohol and polydimethylsiloxane. The amount of emollient(s) in a skin-based topical composition is typically about 5% to about 95%.

Suitable propellants include propane, butane, isobutane, dimethyl ether, carbon dioxide, nitrous oxide, and combinations thereof. The amount of propellant(s) in a topical composition is typically about 0% to about 95%.

Suitable solvents include water, ethyl alcohol, methylene chloride, isopropanol, castor oil, ethylene glycol monoethyl ether, diethylene glycol monobutyl ether, diethylene glycol monoethyl ether, dimethylsulfoxide, dimethyl formamide, tetrahydrofuran, and combinations thereof. Specific solvents include ethyl alcohol and homotopic alcohols. The amount of solvent(s) in a topical composition is typically about 0% to about 95%.

Suitable humectants include glycerin, sorbitol, sodium 2-pyrrolidone-5-carboxylate, soluble collagen, dibutyl phthalate, gelatin, and combinations thereof. Specific humectants include glycerin. The amount of humectant(s) in a topical composition is typically 0% to 95%. The amount of thickener(s) in a topical composition is typically about 0% to about 95%. Suitable powders include beta-cyclodextrins, hydroxypropyl cyclodextrins, chalk, talc, fullers earth, kaolin, starch, gums, colloidal silicon dioxide, sodium polyacrylate, tetra alkyl ammonium smectites, trialkyl aryl ammonium smectites, chemically-modified magnesium aluminum silicate, organically-modified Montmorillonite clay, hydrated aluminum silicate, fumed silica, carboxyvinyl polymer, sodium carboxymethyl cellulose, ethylene glycol monostearate, and combinations thereof. The amount of powder(s) in a topical composition is typically 0% to 95%. The amount of fragrance in a topical composition is typically about 0% to about 0.5%, particularly, about 0.001% to about 0.1%. Suitable pH adjusting additives include HCl or NaOH in amounts sufficient to adjust the pH of a topical pharmaceutical composition.

### Methods of Treatment

The disclosed compositions may be used to modulate glucose levels in a subject. The method may comprise administering a therapeutically effective amount of a pharmaceutical composition comprising glucagon or a glucagon analogue encapsulated within a hydrogel, as described herein, to a subject in need thereof. In various instances, the subject in need thereof may have an insulin disorder, such as diabetes. In various instances, the subject in need thereof may be experiencing a hypoglycemic event. When the subject is hypoglycemic, the glucagon or glucagon analogue may be released from the hydrogel, however, conversely, when the subject is normoglycemic or hyperglycemic, the glucagon or glucagon analogue will not be released from the hydrogel.

In various instances, the therapeutically effective amount comprises 0.1-10 mg. In some instances, the therapeutically effective amount comprises 0.2-9.8 mg; 0.5-9.5 mg; 1.0-9.0 mg; 2.0-8.0 mg; 3.0-7.0 mg; or 4.0-6.0 mg. In some instances, the therapeutically amount comprises no greater than 10 mg; no greater than 9.0 mg; no greater than 8.0 mg; no greater than 7.0 mg; no greater than 6.0 mg; no greater than 5.0 mg; no greater than 4.0 mg; no greater than 3.0 mg; no greater than 2.0 mg; no greater than 1.0 mg; no greater than 0.5 mg; or no greater than 0.2 mg; or no greater than 0.1 mg. In some instances, the therapeutically amount comprises no less than 0.1 mg; no less than 0.2 mg; no less than 0.5 mg; no less than 1.0 mg; no less than 2.0 mg; no less than 3.0 mg; no less than 4.0 mg; no less than 5.0 mg; no less than 6.0 mg; no less than 7.0 mg; no less than 8.0 mg; no less than 9.0 mg; or no less than 10 mg.

In various instances, following administration of the pharmaceutical composition, the subject has blood glucose levels of about 60-110 mg/dL. In various instances, following administration of the pharmaceutical composition, the subject has blood glucose levels of about 65-105 mg/dL; about 70-100 mg/dL; about 75-95 mg/dL; or about 80-90 mg/dL. In various instances, following administration of the pharmaceutical composition, the subject has blood glucose levels of no greater than about 110 mg/dL; no greater than about 100 mg/dL; no greater than about 90 mg/dL; no greater than about 80 mg/dL; no greater than about 70 mg/dL; or no greater than about 60 mg/dL.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All of the various embodiments, aspects, and options disclosed herein can be combined in any variations or iterations. The scope of the compositions, formulations, methods, and processes described herein include all actual or potential combinations of embodiments, aspects, options, examples, and preferences herein described. The exemplary compositions and formulations described herein may omit any component, substitute any component disclosed herein, or include any component disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. All patents and publications cited herein are incorporated by reference herein for the specific teachings thereof.

### EXAMPLES

### Example 1

### Peptide Synthesis and Purification

Peptide amphiphiles were synthesized by solid-phase methods using a CEM Liberty Blue automated synthesizer. See Table 1. Rink amide resin (0.89 emq/g, 100-200 mesh) and Fmoc-protected amino acids were purchased from ChemImpex. Fmoc removal was achieved using 20% piperidine in DMF, with couplings conducted under microwave heating using diisopropylcarbodiimide (DIC) and Oxyma in DMF. Peptides were cleaved from resin and protecting groups were removed by agitation in trifluoroacetic acid (TFA)/triisopropylsilane/H₂O (95:2.5:2.5, v/v/v) for 2 h at room temperature. The solution was evaporated under vacuum to remove most TFA and the product was recovered by precipitating in cold diethyl ether and collected by centrifugation. A solid white powder was air-dried overnight. Peptide purification was next performed on a Biotage Isolera system. The fully dried sample was dissolved in hexafluoro-2-propanol (HFIP) at a concentration of 100-150 mg/mL and injected onto a reversed-phase bio-C18 flash cartridge (50 g) at a flow rate of 40 mL/min with the linear gradient from 0-100% (v/v) acetonitrile (+0.1% NH₄OH) in water. UV absorbance was monitored at 220 and 280 nm for fraction collection. The purified sample was collected, and the purity was verified by electrospray ionization mass spectrometry (ESI-MS, Advion) and analytical HPLC on a C18 Gemini (Phenomenex) column. The purified fractions were lyophilized, yielding a white powder product.

TABLE 1
Peptide Structures

C₁₆—V₂A₂E₂ SEQ ID NO: 1

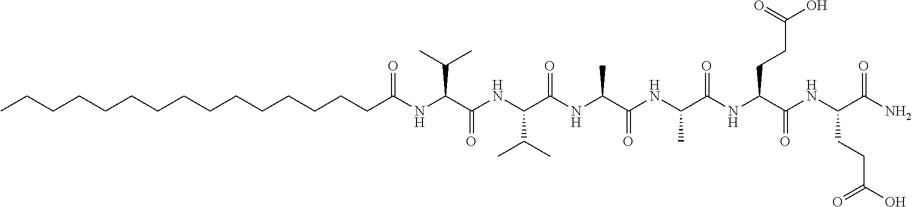 [SMILES: CCCCCCCCCCCCCCCC(=O)N[C@H](C(=O)N[C@H](C(=O)N[C@@H](C)C(=O)N[C@@H](C)C(=O)N[C@@H](CCC(=O)O)C(=O)N[C@@H](CCC(=O)O)C(N)=O)C(C)C)C(C)C]

C₁₆—VA₃E₂ SEQ ID NO: 2

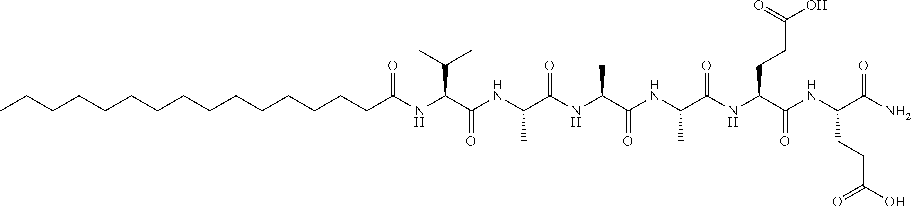 [SMILES: CCCCCCCCCCCCCCCC(=O)N[C@H](C(=O)N[C@@H](C)C(=O)N[C@@H](C)C(=O)N[C@@H](C)C(=O)N[C@@H](CCC(=O)O)C(=O)N[C@@H](CCC(=O)O)C(N)=O)C(C)C]

C₁₆—A₂V₂E₂ SEQ ID NO: 3

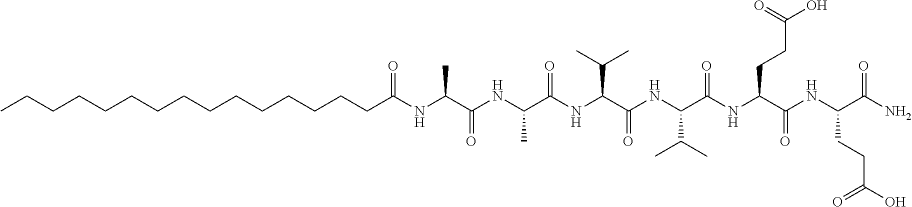 [SMILES: CCCCCCCCCCCCCCCC(=O)N[C@@H](C)C(=O)N[C@@H](C)C(=O)N[C@H](C(=O)N[C@H](C(=O)N[C@@H](CCC(=O)O)C(=O)N[C@@H](CCC(=O)O)C(N)=O)C(C)C)C(C)C]

C₁₀—V₂A₂E₂ SEQ ID NO: 4

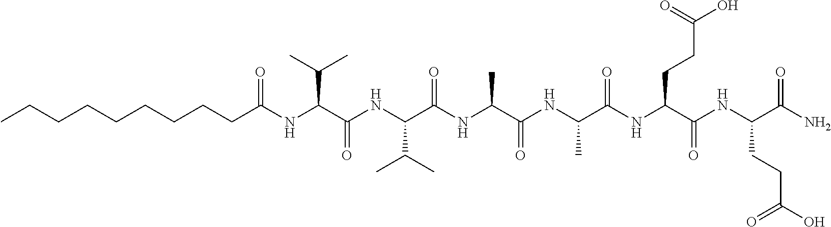 [SMILES: CCCCCCCCCC(=O)N[C@H](C(=O)N[C@H](C(=O)N[C@@H](C)C(=O)N[C@@H](C)C(=O)N[C@@H](CCC(=O)O)C(=O)N[C@@H](CCC(=O)O)C(N)=O)C(C)C)C(C)C]

C₁₀—VA₃E₂ SEQ ID NO: 5

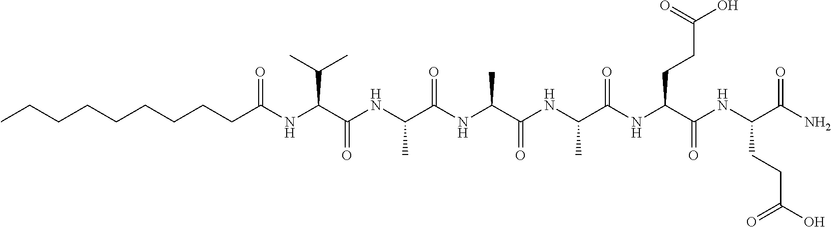 [SMILES: CCCCCCCCCC(=O)N[C@H](C(=O)N[C@@H](C)C(=O)N[C@@H](C)C(=O)N[C@@H](C)C(=O)N[C@@H](CCC(=O)O)C(=O)N[C@@H](CCC(=O)O)C(N)=O)C(C)C]

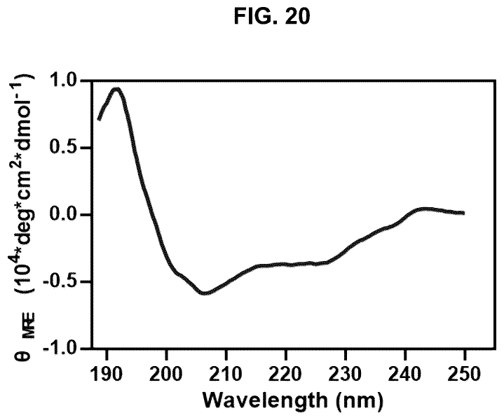

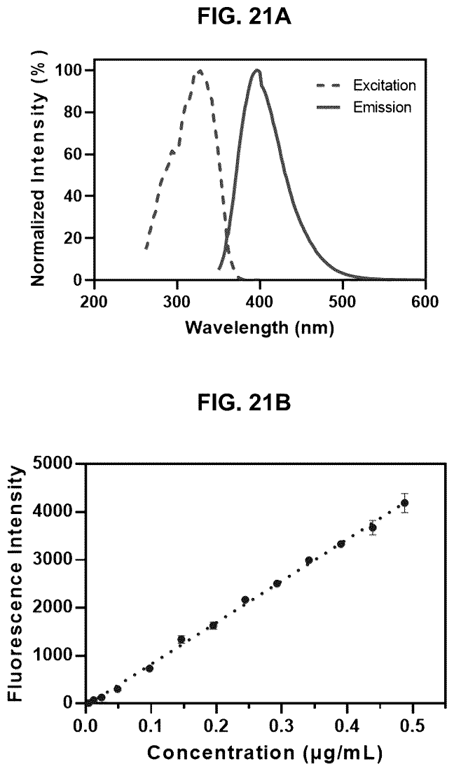

The stable modified glucagon analogue, known as dasiglucagon (Zegalogue®, Zealand Pharma A/S; HSQGTFTSDYSKYLDXARAEEFVKWLEST, where X is 2-aminoisobutyric acid (Aib); SEQ ID NO: 6), and a fluorescent dasiglucagon variant modified with methoxycoumarin (MCA-dasiglucagon) were synthesized and purified according to similar methods as detailed above. To prepare a fluorescent MCA-dasiglucagon, MCA-lysine (Sigma) was inserted in place of the tryptophan residue at position 25 (SEQ ID NO: 7). Both products were verified by ESI-MS and analytical HPLC (data not shown). The native conformation of dasiglucagon was verified by circular dichroism spectroscopy (FIG. 20). The fluorescent properties of MCA-dasiglucagon were verified spectroscopically (FIG. 21).

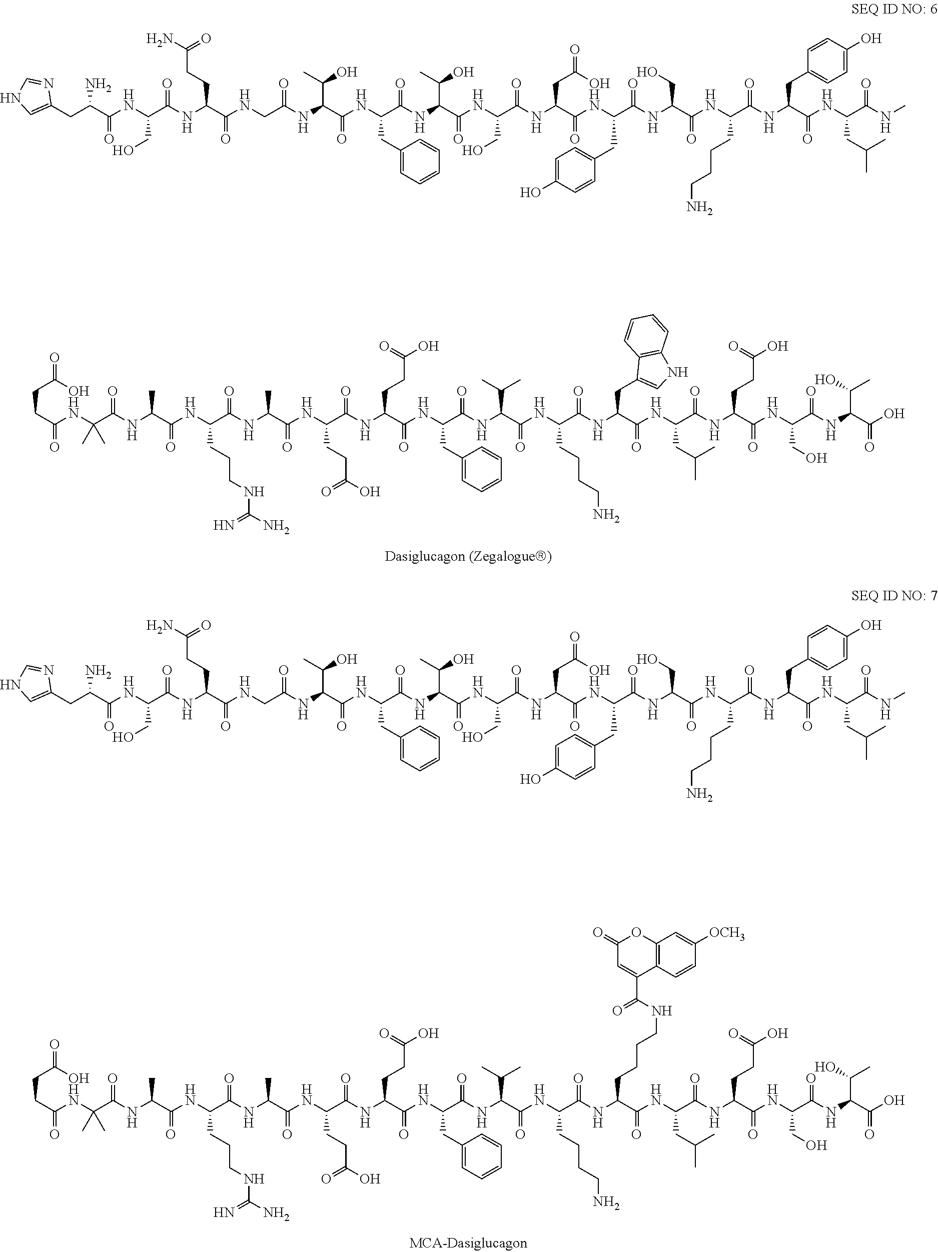

MCA-Dasiglucagon contains a W25K-MCA substitution to accommodate the methoxycoumarin fluorophore modification.
pH-Dependent Self-Assembly and Hydrogelation

The C₁₀-V₂A₂E₂ peptide (SEQ ID NO: 4) amphiphile was first dissolved in DI water at a concentration of 2% (w/v). The solution was adjusted to pH 7.4 using 0.1 M HCl, and then mixed with an equal volume of citrate-phosphate buffer (150 mM buffer+150 mM NaCl) at different pH (4, 5, 6, 7, 8) to form a gel at a concentration of 1% (w/v) in a final buffer concentration of 150 mM.

### Glucose-Dependent Self-Assembly and Hydrogelation

The C₁₀-V₂A₂E₂ peptide amphiphile was first dissolved in DI water at a concentration of 2% (w/v). The solution was adjusted to pH 7.4 using 0.1 M HCl, and then mixed with an equal volume of 300 mM NaCl solution containing 440 U/mL GOₓ and different glucose concentration (0, 100, 200, 300, 400 mg/dL) to achieve final glucose concentrations of 0, 50, 100, 150, and 200 mg/dL and final peptide concentration of 1% (w/v).

### Rheological Characterization

Dynamic oscillatory rheology was performed on a TA Instruments Discovery HR-2 rheometer fitted with a Peltier stage using a parallel plate geometry with diameter of 25 mm to test the mechanical properties of all peptide hydrogels. Samples were prepared at a concentration of 1% (w/v) in buffers of different pH or glucose concentration, as described above, and measured 24 h after gel preparation. An amplitude sweep was first performed to determine the linear viscoelastic range for each hydrogel condition, and then a frequency sweep was performed at constant strain. Subsequently, a time-sweep (1% strain, 10 rad/s angular frequency) was performed for all hydrogels to measure and compare the storage modulus (G′). A step-strain study alternating between 1% strain for 2 min and 100% strain for 30 s at angular frequency 10 rad/s was also performed for the pH 5 hydrogel.

### Circular Dichroism Spectroscopy

Near-UV circular dichroism spectroscopy (CD) was performed using a Jasco J-815 instrument. Samples were typically prepared at a concentration of 0.1% (w/v) in 50 mM phosphate buffer at various pH (5-8) and 50 μL peptides solution was transferred to a quartz plate cuvette with pathlength of 0.1 mm. Three spectra were collected (range of 250-185 nm, 50 nm/min scanning speed) and averaged for each sample. For quality control, spectra were truncated upon photomultiplier voltage (HT) exceeding 600 mV.

### FTIR Characterization

The peptide was first dissolved in D₂O at a concentration of 2% (w/v). The solution was adjusted to pH 7.4 using 0.1 M deuterium chloride (DCI), and then mixed with an equal volume of citrate-phosphate buffer prepared in D₂O (150 mM buffer+150 mM NaCl) at different pH (5, 6, 7, 8) to form a gel at a concentration of 1% (w/v) in a final buffer concentration of 150 mM. Gel samples of 10 μL for each pH were dropped, dried for 10 minutes, and analyzed using a Jasco FT/IR-6300 spectrometer. A background of the buffer in D₂O was subtracted from all spectra.

### Thioflavin T Assay

Peptide solutions at concentration of 0.1% (w/v) were prepared in citrate-phosphate buffer (15 mM buffer+150 mM NaCl) at pH value of 5 and 7 separately. Subsequently, 198 μL of these peptide solutions were combined with 2 μL of a 10 mM thioflavin T (ThT) stock. Fluorescence was measured on a Tecan M200 plate reader (Ex: 485 nm, Em: 528 nm) every 15 s. Following 300 s of equilibration, 2 μL 1 M NaOH was added into pH 5 peptide solution reaching a final pH value of 7. For pH 7 peptide solution, 2 μL DI water was added. The volume of NaOH was verified to achieve pH 7 in this buffer system. The change of fluorescence intensity for both solutions was immediately recorded over another 300 s. A background of ThT in pH 5 and pH 7 buffer was subtracted from all spectra.

### Transmission Electron Microscopy

Transmission electron microscopy (TEM) was performed using a JEOL 2011 instrument. Peptide samples were prepared at a concentration of 0.1% (w/v) in a 50 mM phosphate buffer, at various pH (5, 6, 7, 8). These samples were deposited at a volume of 10 μL onto a carbon-coated copper grid (200 mesh), wicked using filter paper after 30 s, and stained using 5 μL of a uranyl acetate solution. Grids were dried overnight prior to imaging.

### Scanning Electron Microscopy

Peptide hydrogel samples were prepared at a concentration of 1% (w/v) in pH 5 citrate-phosphate buffer and fixed in a 4% glutaraldehyde solution in the same buffer at 4° C. overnight. Fixed peptide samples were washed in citrate phosphate buffer and DI water and serially dehydrated in 35%, 50%, 70%, 95%, and 100% ethanol. The dehydrated samples were dried using an Autosamdri®-931 CO₂ critical point dryer (Tousimis, Rockville, MD, USA). The dried samples were sputter-coated with a 3 nm Iridium layer and imaged using a FEI Magellan 400 field-emission scanning electron microscope at an accelerating voltage of 2 kV.

### Glucose-Dependent Glucagon Release

To evaluate glucose-responsive dasiglucagon release, 100 μL peptide hydrogels (1% w/v) containing 11 U GOₓ, 38.84 U catalase, and 0.02 mg MCA-dasiglucagon were prepared in pH 5 citrate-phosphate buffer. Once formed, the hydrogels were incubated within 6-well plates in 4 mL of either pH 5 or pH 7.4 buffer for pH-dependent release, or in a pH 7.4 buffer containing 0, 50 100, 150, 200 mg/dL glucose for glucose-dependent release. At each time point, a 20 μL aliquot was taken for fluorescence analysis (Ex: 322 nm, Em: 398 nm) with the MCA-dasiglucagon concentration determined using a standard curve (FIG. 21B). As glucose was continually consumed during the study, 20 μL of concentrated glucose solution was added to maintain a constant glucose level as verified by readings using a handheld glucometer. After 24 h, all samples were treated with 0.1 M NaOH to disrupt any remaining hydrogel structure and fluorescence of residual MCA-dasiglucagon was measured to ensure mass balance closure. The pH of the bulk release buffer was also measured using a pH meter. To evaluate the impact of a sudden drop in glucose level on release, a 50 μL hydrogel containing 5.5 U GOₓ, 19.42 U Catalase, and 0.01 mg MCA-dasiglucagon was incubated in 2 mL pH 7.4 buffer containing 100 mg/dL glucose for 2 h. Subsequently, the incubation solution was removed and replaced with the same volume of pH 7.4 buffer containing no glucose.

### Blood Glucose Control In Vivo

To assess the ability of this technology to act in a preventative role to limit the onset and severity of hypoglycemia, a mouse model was established. Male C57BL6/J mice, aged 8 weeks, were induced to be diabetic by the destruction of pancreatic β-cells using a single intraperitoneal (i.p.) injection of streptozotocin (STZ, Cayman Chemical) at a dose of 150 mg/kg, according to published dosing protocols. It is noted that for the dosing of all compounds and agents here, mice were assumed to have a body weight of 25 g. Insulin-deficient diabetes was verified at 9-13 days following STZ treatment using handheld blood glucose meters (CVS brand) to ensure unfasted blood glucose (BG) levels of 600+ mg/dL. These glucometers measure blood glucose values in the range of 20-600 mg/dL. For convention in presenting data, values of “high” are plotted as 600 mg/dL, while values of “low” are plotted as 20 mg/dL.

Mice were fasted overnight for a period of 9 h, after which time BG was again measured. Mice having a fasted BG<450 mg/dL were triaged and removed from further study. Remaining mice were dosed with basal insulin detemir (Levemir®, Novo Nordisk) via subcutaneous (s.c.) injection at a dose of 0.5 IU/kg in a total injection volume of 100 μL. Following an additional 4 h fast to normalize blood glucose levels to a normal range for mice (˜180 mg/dL), mice were randomized into groups and treated with buffer, dasiglucagon alone (0.01 mg), or the full peptide amphiphile (PA) hydrogel system at pH 5 and 1% w/v loaded with 0.01 mg dasiglucagon, 1.1 U GOₓ, 7.77 U Catalase in a 50 μL s.c. injection. This material formulation was verified to be stable following extrusion through a syringe into a pH 7 buffer containing 100 mg/dL glucose. Blood glucose was monitored serially following treatment, which for purposes of data visualization was set as t=0 min. At 2 h following administration of treatments, hypoglycemia was induced by i.p. injection of AOF recombinant human insulin (Gibco) at a dose of ˜2.5 IU/kg in 100 μL of saline. BG levels were monitored for an additional 3 h after insulin overdose to monitor the onset of, and recovery from, hypoglycemia. Mice exhibiting “low” readings, as well as those which died from hypoglycemia, were noted with BG values of 20 mg/dL. In total n=9 mice per group were assessed by these methods, performing all treatments in two separate experiments on different mouse cohorts at different times and combining the data for analysis here. Mice were fasted for the duration of the study but had continuous access to water. These studies were detailed in a protocol approved by the University of Notre Dame Animal Care and Use Committee and adhered to all relevant Institutional, State, and Federal guidelines. Statistical testing between treatment and control groups was performed using one-way ANOVA with Tukey multiple comparison post-hoc testing (GraphPad Prism v9.0).

### Example 2

### Peptide Design

Peptides constitute a class of molecules extensively explored as supramolecular materials for biomedical applications. To prepare a pH-responsive gelator and achieve self-assembly governed by consumption of glucose fuel (FIG. 2A-B), the peptide amphiphile (PA) platform was explored here. This supramolecular motif typically combines a hydrophobic directive for assembly in water from a saturated alkyl chain appended at a terminal position with a peptide sequence consisting of residues for lateral association through β-sheet hydrogen bonding as well as residues bearing charged groups to enhance solubility and amphiphilicity of the molecule. These molecules can self-assemble in water to form high aspect-ratio nanofibrils templated by hydrogen bonding along the long axis of the fiber, and further physically entangle to form percolated hydrogel networks. The interplay of attractive and repulsive forces entailed in this molecular design gives rise to a tunable extent of molecular cohesion, and can be varied by altering tail length, β-sheet sequence, or the number/identity of charged residues. In particular, the electrostatic repulsion between charge-bearing hydrophilic amino acids can be modulated by addition of counterions or by changing pH relative to the pKa of charged residues so as to induce self-assembly, stabilize nanofibrils, and increase the extent of physical crosslinking in a resulting hydrogel.

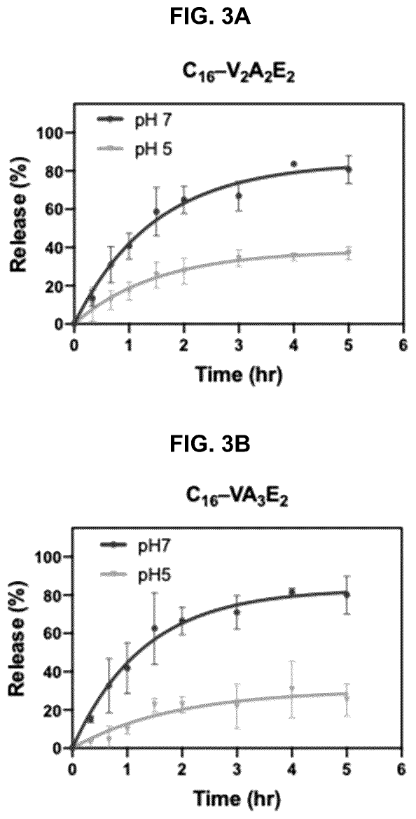

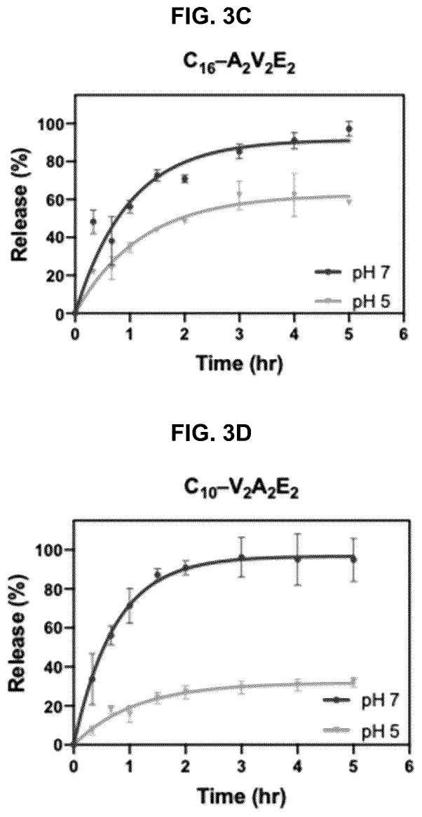

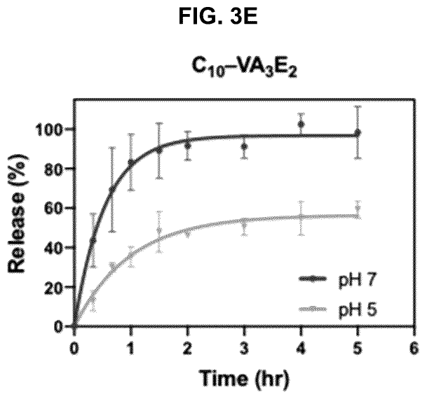

To interface a supramolecular PA gelator with glucose-fueled assembly directed by the enzymatic actuation of GOₓ, a variant was desired to form stable hydrogels in the acidic microenvironment that would result locally from conversion of a normal level of glucose, but which transitioned to a soluble molecule under glucose-limited conditions. GOₓ conversion of physiological levels of glucose to yield gluconic acid can lead to a microenvironmental pH in the range of ˜4-5.6. Accordingly, a molecular design was envisioned bearing the typical saturated alkyl chain and β-sheet-forming sequence coupled to glutamic acid as the hydrophilic domain; the pKa of its carboxylate R-group (˜4.5) can shift upward in the range of 4.5-6 due to aggregation effects from PA self-assembly. Accordingly, glutamic acid (E) should be significantly protonated (uncharged) at acidic pH levels achieved by GOₓ in normal glucose levels, yet deprotonated (negatively charged) at physiological pH. Different PA sequences were synthesized to vary the alkyl tail length (C₁₀ and C₁₆) and valine (V)-alanine (A) β-sheet sequence (V₂A₂, VA₃, A₂) with a conserved hydrophilic head group (E₂). The intention of screening this small set of molecules was to probe pH-responsive release to determine the optimal balance of attractive and repulsive forces required for the envisioned application of assembly at normal glucose (i.e., low pH actuated by GOₓ) but disassembly and glucagon release in low glucose conditions (i.e., neutral-buffered physiological pH). Based on a preliminary screen comparing release rates of a model macromolecule at pH 5 and 7 (FIG. 3A-E), a final sequence of C₁₀-V₂A₂E₂ (FIG. 2C) was selected for further evaluation. The selected sequence has a shorter C₁₀ alkyl segment than commonly used in most reported PA materials. The V₂A₂ sequence is thought to be effective for β-sheet hydrogen bonding, while still being shorter than most commonly explored sequences. The glutamic acid residues then afford pH sensitivity over the range desirable for GOₓ. Thus, a reduction in cohesive forces from a shorter alkyl segment and a 4-residue β-sheet forming segment was thought to enable more rapid responsiveness dependent on the charge state of the glutamic acid residues.

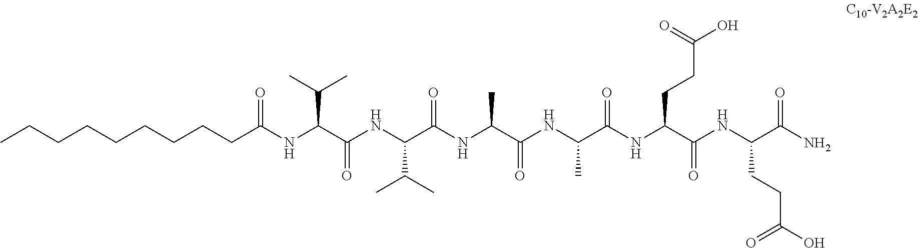 [SMILES: CCCCCCCCCC(=O)N[C@H](C(=O)N[C@H](C(=O)N[C@@H](C)C(=O)N[C@@H](C)C(=O)N[C@@H](CCC(=O)O)C(=O)N[C@@H](CCC(=O)O)C(N)=O)C(C)C)C(C)C]

pH-Dependent Self-Assembly and Hydrogelation

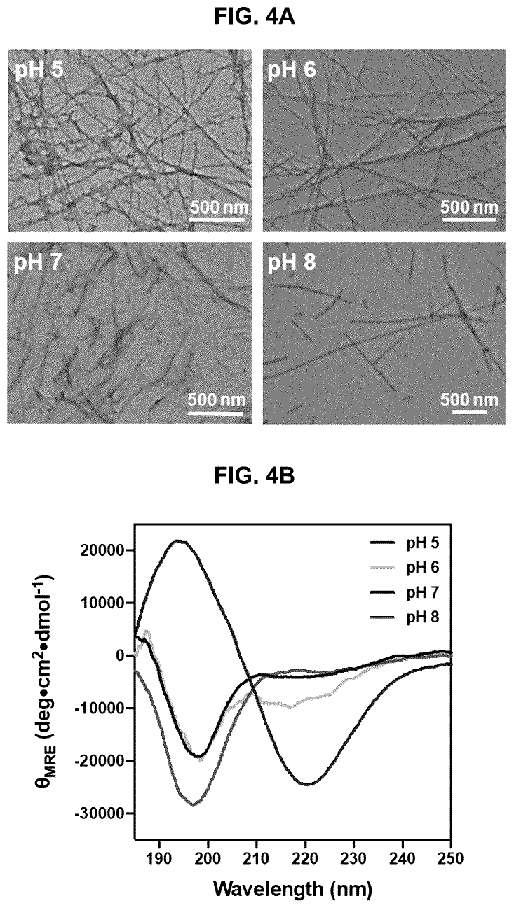

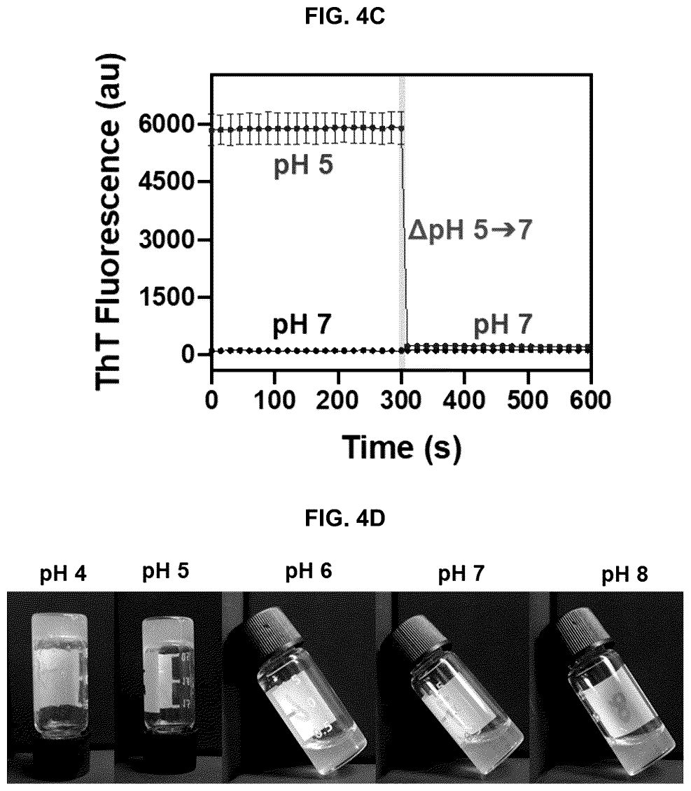

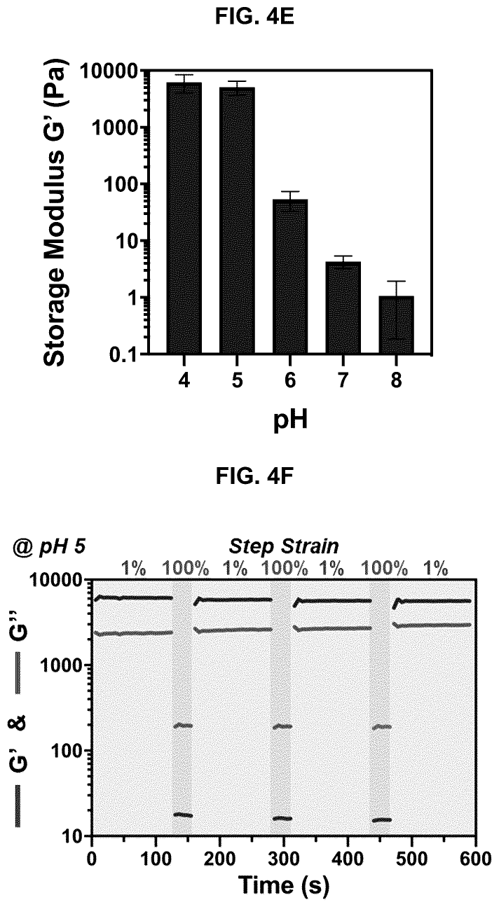

Given the importance of pH to the envisioned mechanism of glucose-fueled assembly, pH-responsive self-assembly of lead PA C₁₀-V₂A₂E₂ was first characterized. Samples were prepared in buffers of pH values ranging from 5 to 8, cast as dry films, and imaged with transmission electron microscopy (TEM) (FIG. 4A). It is noted that TEM performed on dry films is subject to artifacts arising from drying and sample concentration on the grid. However, qualitative observations support a general trend for a reduction in nanofibril length as well as reduced nanofibril bundling as pH was increased from 5 to 8. For example, samples at pH 5 had a high density of elongated nanofibrils with substantial bundling, while samples prepared at pH 8 had sparse nanostructure with observed nanofibrils being significantly shorter and exhibiting almost no bundling.

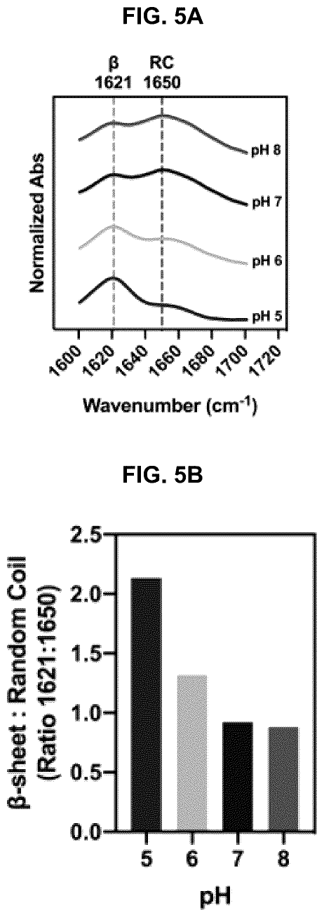

Near ultraviolet circular dichroism (CD) spectroscopy was performed to characterize amino acid secondary structure in buffers of various pH (FIG. 4B). CD is frequently used to qualitatively assess the extent of β-sheet hydrogen bonding in PA materials. Spectra collected for C₁₀-V₂A₂E₂ at pH 5 exhibited a characteristic β-sheet signature with a negative peak at 220 nm and positive peak at 194 nm. Meanwhile, the CD spectra for the sample prepared at pH 8 exhibited a negative peak at 197 nm characteristic of a random coil secondary structure. The samples prepared at intermediate pH values of 6 and 7 were primarily random coil, with some evidence of residual β-sheet character for the pH 6 sample. These findings support increased β-sheet cohesion in self-assembled nanofibrils resulting from less electrostatic repulsion in the glutamic acid head group at lower pH. Fourier-Transform Infrared Reflectance (FTIR) spectroscopy was also performed to monitor signals attributed to β-sheet and random coil structures as a function of pH, revealing the same trend of β-sheet reduction and random coil emergence as pH increases (FIG. 5A-B).

Thioflavin-T (ThT) provides another method to study the β-sheet character of peptide self-assemblies, relying on increased fluorescence of this dye when embedded in β-sheet-rich domains. The background-subtracted fluorescence of C₁₀-V₂A₂E₂ at pH 5 and pH 7 was first compared; pH 5 exhibited strong ThT fluorescence indicative of ThT bound to β-sheet-rich structures while limited fluorescence was measured at pH 7 (FIG. 4C). After 300 s incubation, the pH was increased to pH 7 rapidly by adding a small volume of NaOH. ThT fluorescence disappeared by the next reading (˜15 s), indicating immediate loss of β-sheet character upon pH neutralization. Accordingly, pH offers an effective and rapid means to activate and deactivate stabilizing β-sheet structures in C₁₀-V₂A₂E₂ assemblies.

The gelation of C₁₀-V₂A₂E₂ was next studied at 1% w/v in buffers of pH 4 to pH 8 under physiologic salt concentration. Gross visual inspection of PA samples following equilibration at these various pH levels (FIG. 4D) revealed the formation of a stable hydrogel immediately at pH 4 and pH 5. This observation aligns with the predicted pKa of the glutamic acid side-chain. At pH 6, the solution was notably viscous, yet this sample flowed when subjected to vial inversion. Samples prepared at pH 7 and pH 8 were even less viscous and flowed with relative ease upon vial inversion. It is noted that samples of all pH conditions were viscous and not fully translucent, indicating the presence of some nanostructure in all samples.

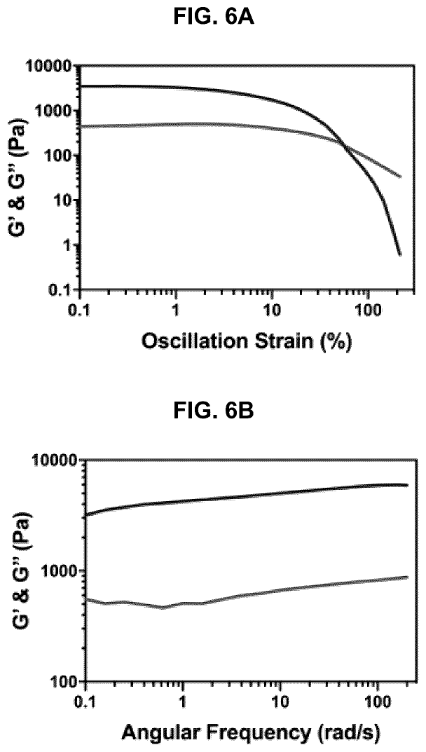

Vial inversion affords a crude means to inspect bulk differences in apparent hydrogelation. By contrast, rheological testing enables quantitative insights into the mechanical and dynamic properties of these materials. Oscillatory rheology was thus performed to quantify the observed pH-dependent differences in hydrogelation. Samples were first assessed with a strain sweep to determine the linear viscoelastic range, and then gel-forming samples were subjected to a frequency sweep to verify the range of oscillatory frequency rates wherein the storage modulus (G′) exceeded the viscous/loss modulus (G″). As an example, a sample that formed a mechanically robust hydrogel in buffer at pH 5 exhibited linear behavior with G′>G″ when exposed to strains in the range of ˜0.1-5%, with the hydrogel being mechanically compromised at a critical strain of ˜55% (FIG. 6A-B). This sample also exhibited scarce frequency-dependent G′ behavior, with G′ values ˜10× higher than G″ over the full range of frequency assessed. This behavior in a frequency sweep is indicative of relaxation times for physical interactions in the hydrogel network, such as nanofiber bundling and intersection, being significantly slower than the range of frequencies probed in dynamic rheological testing.

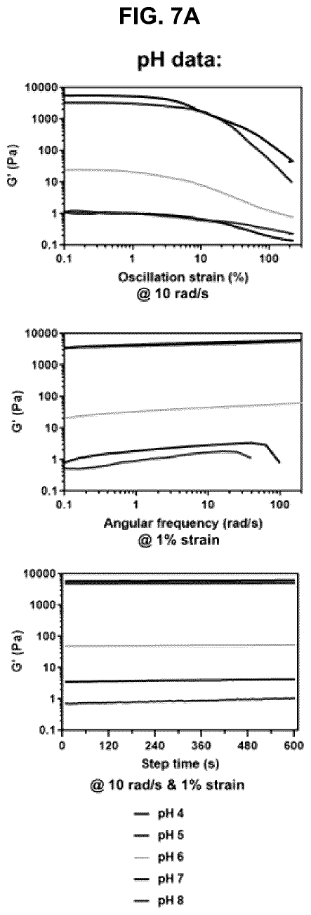

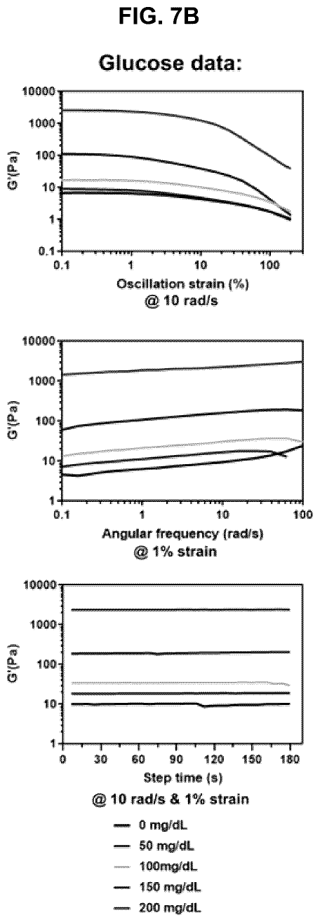

The G′ values for samples at each pH were compared at a constant strain of 1% and frequency of 10 rad/s. When comparing G′ values across the full range of pH explored (n=2 gels/sample), pH-dependent mechanical properties were clearly evident (FIG. 4E, FIG. 7). G′ values were found to decrease with increasing pH as follows: 6.2 kPa (pH 4), 5.1 kPa (pH 5), 53.7 Pa (pH 6), 4.3 Pa (pH 7), and 1.2 Pa (pH 8). Limited differences were observed for G′ in samples of pH 4 and pH 5, yet G′ was reduced by two orders of magnitude for samples prepared at pH 6 and another order of magnitude for samples prepared at pH 7. Samples prepared at pH 8 had G′ measurements at the lower limit of instrument sensitivity.

For peptide-based gelators, the magnitude of G′ is correlated with an increase in the length, stiffness, and extent of bundling for high aspect-ratio nanostructures in the hydrogel. An increased propensity for β-sheet hydrogen bonding often underlies these changes in stiffness. Rheological data coupled with evidence from TEM, CD, FTIR, and ThT studies suggests a mechanism whereby the increased negative charge of assemblies at pH levels of 6 and above drives an increase in repulsive forces within nanofibers to reduce packing and β-sheet formation, shorten the overall nanofiber length, and promote lower extents of aggregation. For any structures that do form, electrostatic repulsion between nanofibers due to increased negative charges would act to limit the extent of fiber bundling and physical crosslinking. Accordingly, from these comparative rheological results, along with evidence from TEM, CD, FTIR, and ThT, it can be inferred that glutamic acid residues in the assembled structures have a likely pKa in the range of 5-6, thus leading to the dramatic shift in pH-dependent G′ observed when transitioning between these two pH levels.

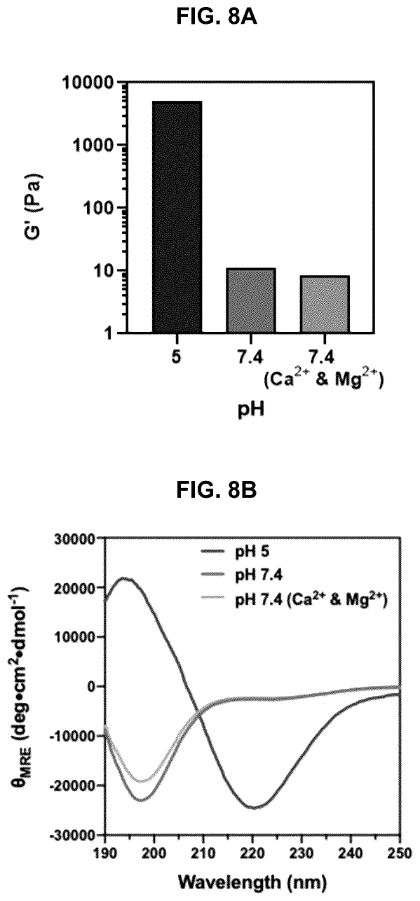

Rheological testing under cyclic strain is typically performed to assess the self-healing capacity of a physically crosslinked hydrogel in the context of its suitability for injection-based applications. Here, the PA hydrogel prepared at pH 5 was subjected to step-strain cycling between 1% and 100% strain at a constant frequency of 10 rad/s (FIG. 4F). This high strain was selected to be in excess of the critical strain for the material (G″>G′ at >55% strain). After multiple cycles of 30 s duration at high strain, G′ values for the material were recovered instantly upon a return to low strain. In the context of the eventual envisioned application of these materials, a stable hydrogel containing GOₓ could be administered in a pH 5 buffer through a syringe into a normoglycemic environment and recover its mechanical properties nearly instantly in situ prior to being placed under assembly control by consumption of physiological glucose fuel. Divalent cations like Ca²⁺ and Mg²⁺ are known to stiffen PA hydrogels of this type via ionic crosslinking of glutamic acids. To assess whether such an effect would compromise pH-responsive properties in vivo by stabilizing nanofibers at neutral pH, CD and rheology were performed under physiological Ca²⁺ and Mg²⁺ levels. No change in the β-sheet content or storage modulus of the material was found at pH 7.4 upon introduction of these divalent ions (FIG. 8A-B).

### Glucose-Dependent Gelation

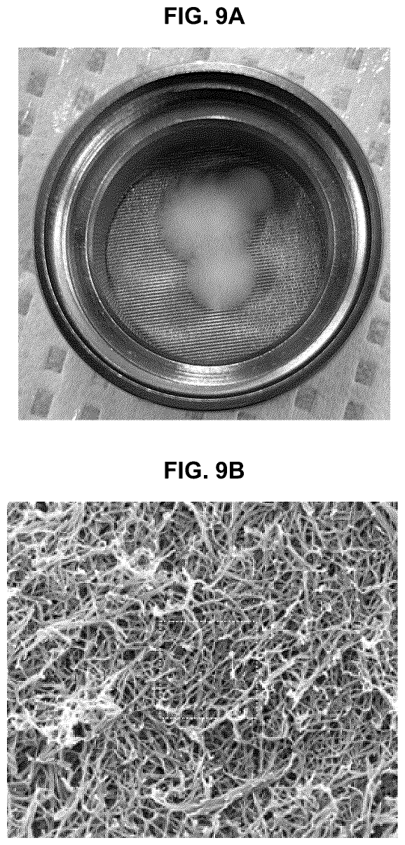

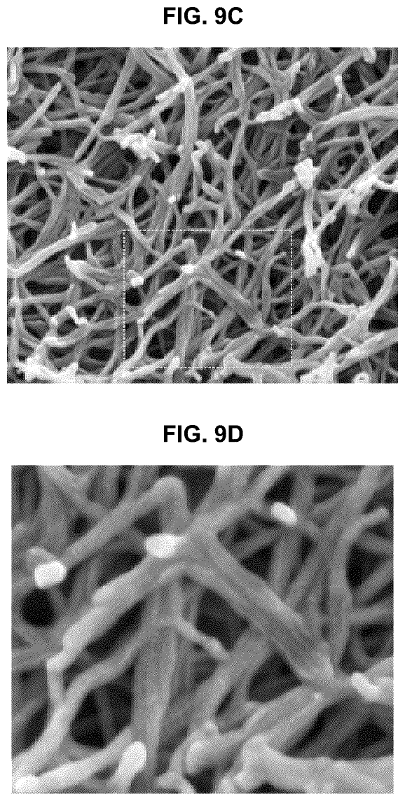

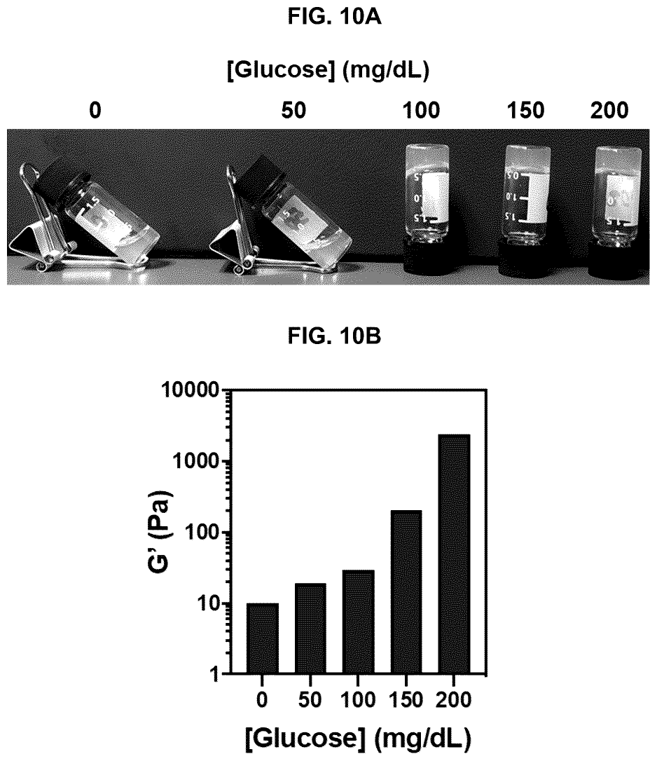

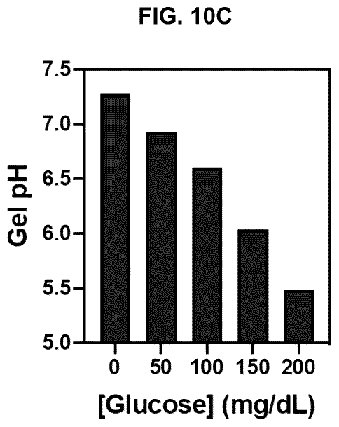

After pH-dependent self-assembly and gelation was established, glucose-dependent gelation was explored through the inclusion of GOₓ to actuate glucose into a pH stimulus. One benefit of hydrogelation of this material at 1% w/v is the significant hydrated and interconnected porosity (FIG. 9A-D) to enable encapsulation of proteins like GOₓ to drive gelation in response to glucose. Samples of C₁₀-V₂A₂E₂ were prepared at 1% w/v in buffer at pH 7.4, with glucose added to achieve concentrations ranging from 0 to 200 mg/dL (0 to 11.1 mM). These ranges of glucose were selected to span typical physiological glucose levels in the healthy state and extend down to levels corresponding to hypoglycemia. Following equilibration for 24 h, samples were assessed grossly for gelation by vial inversion (FIG. 10A), as performed in pH-dependent gelation studies. Visual inspection following vial inversion revealed self-supporting hydrogels at glucose concentrations of 200 mg/dL and 150 mg/dL. The material prepared with 100 mg/dL glucose was very viscous but slowly flowed upon inversion, while those prepared at 0 mg/dL and 50 mg/dL were not self-supported on inversion.

Rheological studies were performed to quantify differences in hydrogel character arising from glucose (FIG. 10B). Measurements were collected as before for gels prepared in each of the different glucose-containing buffers. The value of G′ increased with increasing levels of glucose, as follows: 10.2 Pa (0 mg/dL), 19.0 Pa (50 mg/dL), 29.5 Pa (100 mg/dL), 206.6 Pa (150 mg/dL), and 2.4 kPa (200 mg/dL). Given results of pH-dependent gelation for C₁₀-V₂A₂E₂ PA, it was expected that inclusion of GOₓ would actuate increased glucose level into a local reduction in pH driving hydrogelation. A microelectrode pH probe was submerged within each hydrogel (FIG. 10C), and glucose-dependent pH reduction was recorded, ranging from pH 7.3 (0 mg/dL) to pH 5.5 (200 mg/dL). It is noted that all samples began at pH 7.4; enzymatic conversion of glucose afforded by GOₓ was thus responsible for reducing pH of the fluid within the hydrogel. These measured pH values support findings from rheology as higher G′ was observed at higher glucose concentration which translated to a lower sampled pH. In addition, these findings also confirmed results for the relationship between G′ and pH, with similar results obtained for comparable pH values realized upon addition of glucose. Small discrepancies in G′ values between the pH and glucose studies presented here may be attributed to inclusion of the large GOₓ protein (160 kDa) within the nanofibrillar mesh of the hydrogel. The fixed and finite glucose concentrations within these small volume hydrogels may somewhat limit direct comparisons with concentrations for their use in a physiological setting, as a replenishable glucose supply could further stiffen the materials through enhanced pH reduction.

The results of these glucose-dependent gelation studies support this platform coupled with GOₓ to translate glucose levels into changes in nanoscale and bulk material properties. Based on common interpretations of G′ values for nanofibrillar peptide hydrogels, higher glucose concentrations here corresponded to a more highly interconnected network arising from physical interactions between longer and stiffer nanostructures. As glucose levels were reduced to approach hypoglycemic levels, the network topology as measured by G′ decreased by several orders of magnitude. Thus, the self-assembly of C₁₀-V₂A₂E₂ PA coupled with GOₓ afforded a reliable platform wherein local glucose concentration could be manifest in hydrogel formation and mechanical properties.

### Glucose-Dependent Glucagon Release

After identifying a gelator capable of both pH- and glucose-responsive self-assembly and hydrogelation, controlled release of an encapsulated therapeutic glucagon payload was next assessed. Screening of PA sequences identified the lead sequence on the basis of pH-dependent controlled release of a neutral model macromolecule (3 kDa FITC-dextran, FIGS. 3A-E, Table 2), supporting a mechanism of pH-directed change in network structure dictating the rate of passive release of macromolecules comparable in size to glucagon. For functional release studies, the dasiglucagon analogue was selected as the payload due to its improved solubility and stabilized secondary structure relative to native glucagon. For ease in detection during release studies, a fluorescent methoxycoumarin (MCA) group was substituted in place of a tryptophan on the dasiglucagon sequence. Hydrogels were prepared in all cases in a pH 5 buffer at 1% w/v to ensure initial gelation. Each 100 μL hydrogel also contained GOₓ, catalase, and 0.2 mg of MCA-dasiglucagon. It is noted that catalase was added to these formulations in order to catalyze the conversion of the toxic GOₓ byproduct, H₂O₂, into H₂O and O₂ in advance of in vivo application, as is commonly done to reduce toxicity for other GOₓ-based glucose-responsive materials. Each hydrogel was immersed in 4 mL pH 7.4 buffer containing different physiologically relevant glucose concentrations, while a control hydrogel was immersed in a pH 5 buffer to understand dasiglucagon leakage from the fully stable hydrogel. In order to maintain desired glucose concentrations for each group, a small volume of a concentrated glucose solution was added during release studies to replenish the glucose fuel consumed by GOₓ, enabling stable glucose concentrations over time as confirmed by glucometer readings.

TABLE 2
3 kDa FITC-dextran Release

| Sequence | Δ Release % (pH 7-5 at 5 hr) |
|---|---|
| C₁₆—V₂A₂E₂ | 43.6 |
| C₁₆—VA₃E₂ | 53.8 |
| C₁₆—A₂V₂E₂ | 38.9 |
| C₁₀—V₂A₂E₂ | 62.6 |
| C₁₀—VA₃E₂ | 39.1 |

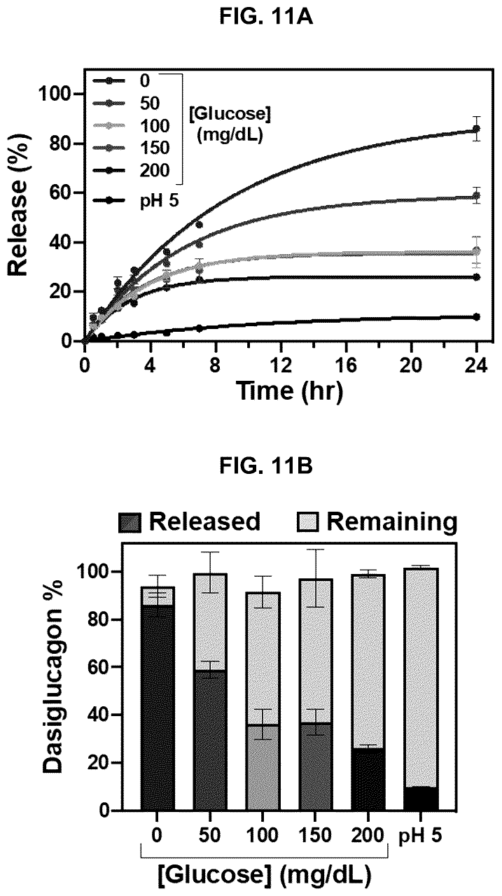

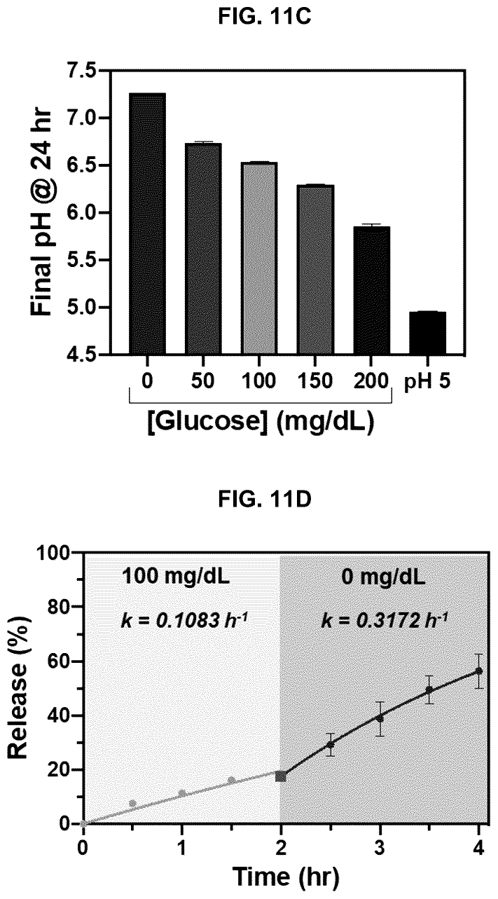

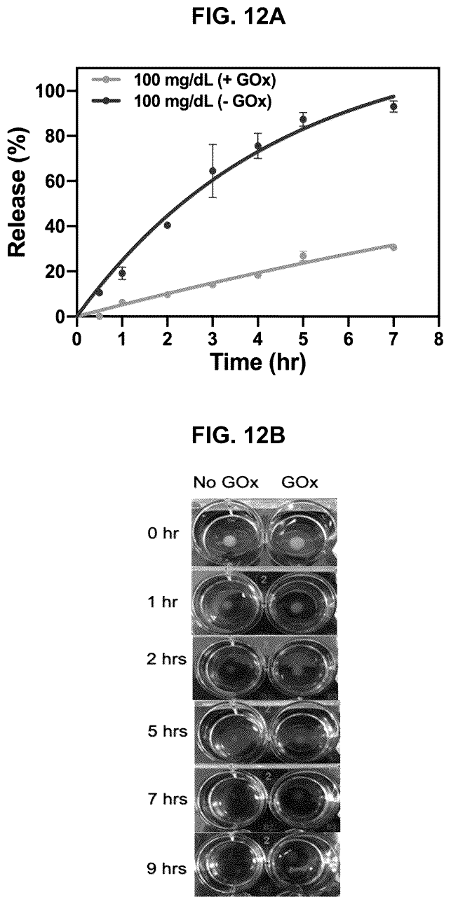

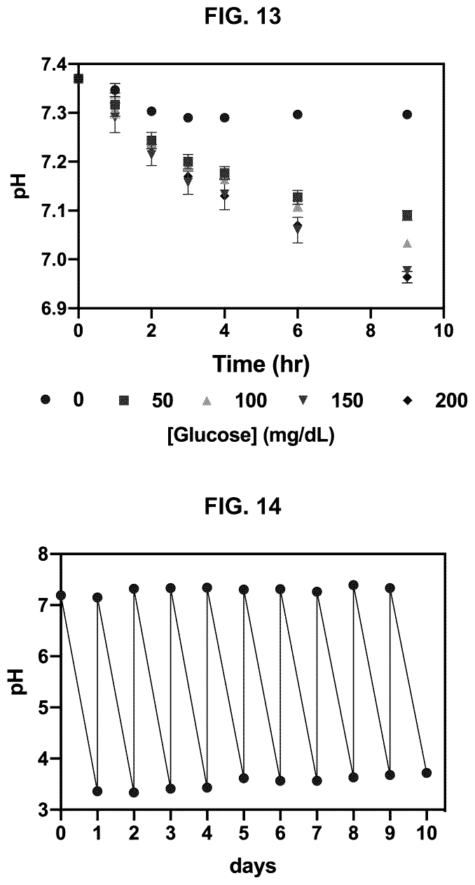

Studying release over a range of glucose concentrations revealed clear glucose-dependent dasiglucagon release (FIG. 11A), with both the rate and amount of dasiglucagon release decreasing with increasing glucose levels in the buffer. At 24 h, retained dasiglucagon in each sample was extracted and quantified to verify mass balance closure (FIG. 11A). When time-dependent release data were fit to a standard first-order model, the model plateau values obtained ranged from 91% (0 mg/dL) to 26% (200 mg/dL), indicating the role of GOₓ conversion of glucose in stabilizing the hydrogel and limiting its release of encapsulated dasiglucagon. The pH 5 control hydrogel, meanwhile, showed slow and limited release with a first-order plateau value of 11%. The inclusion of GOₓ was critical to achieving controlled dasiglucagon release in the presence of glucose (FIG. 12A-B). The pH of the bulk buffer, which began at pH 7.4, decreased at 24 h as a result of acidification from GOₓ action, reaching a pH of 5.9 in the buffer containing 200 mg/dL glucose (FIG. 11B). This reduction in bulk pH occurred steadily over the first several hours of gel incubation (FIG. 13). The initial release rates during the first 3 h of incubation were also higher for samples incubated in 0 mg/dL (12.3%/hr) compared to those in 200 mg/dL glucose (7.3%/hr), supporting glucose-dependent release even at early times before a significant bulk acidification was realized to stabilize the hydrogel and slow release. It is noted that bulk pH measurements do not necessarily reflect the local microenvironmental pH within the hydrogel that may arise from concentration gradients, preferential proton localization, and/or differential pH buffering by charged residues of the material. Local reductions in microenvironmental pH have been postulated as a mechanism for function of many related hydrogel systems that use GOₓ to induce pH-responsive swelling and insulin release. The lag in glucose-induced material stabilization, and concomitant burst release in the initial period, offers an opportunity for future refinement of these and related technologies.

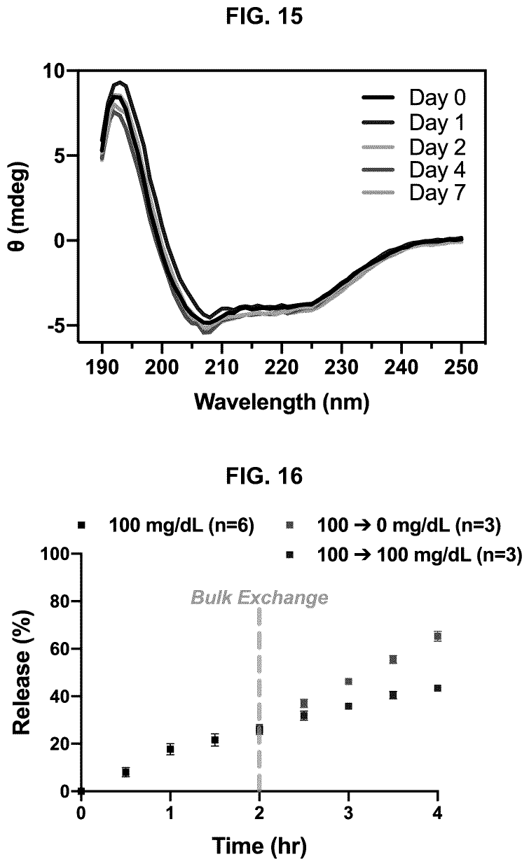

GOₓ activity was preserved within the gel for at least 10 days based on its ability to repeatedly lower pH upon daily recharge with an unbuffered and neutral 200 mg/dL glucose solution (FIG. 14). This is expected given the weeks-long use of GOₓ as a sensor in implanted continuous glucose monitors. Moreover, the dasiglucagon payload remains stable for at least 7 days of incubation in the pH 5 environment expected within the hydrogel, showing no sign of degradation or amyloid formation (FIG. 15).

The envisioned application for glucagon delivery using glucose-stabilized materials is as an administered prophylactic, ready in the event of subsequent onset of a serious hypoglycemic episode. Thus, a rapid reduction in glucose level should trigger material dissipation and accelerated glucagon release. To assess this use, hydrogels prepared identically to those in the release studies were first incubated in a buffer of 100 mg/dL (resembling normoglycemia) and following 2 h the buffer was exchanged to 0 mg/dL (FIG. 11C). The instantaneous reduction in glucose led to an acceleration in dasiglucagon release from 10.8%/hr before the buffer change to 31.7%/hr after. In this case, release when the sample was switched to 0 mg/dL glucose was even faster than it was in the case where the experiment was initialized from 0 mg/dL glucose buffer. Complete buffer exchange can increase release due to dilution, swelling, and/or gel erosion, but this alone did not account for the more rapid release following introduction of the glucose-free buffer (FIG. 16).

### Hypoglycemia Prevention in Diabetic Mice

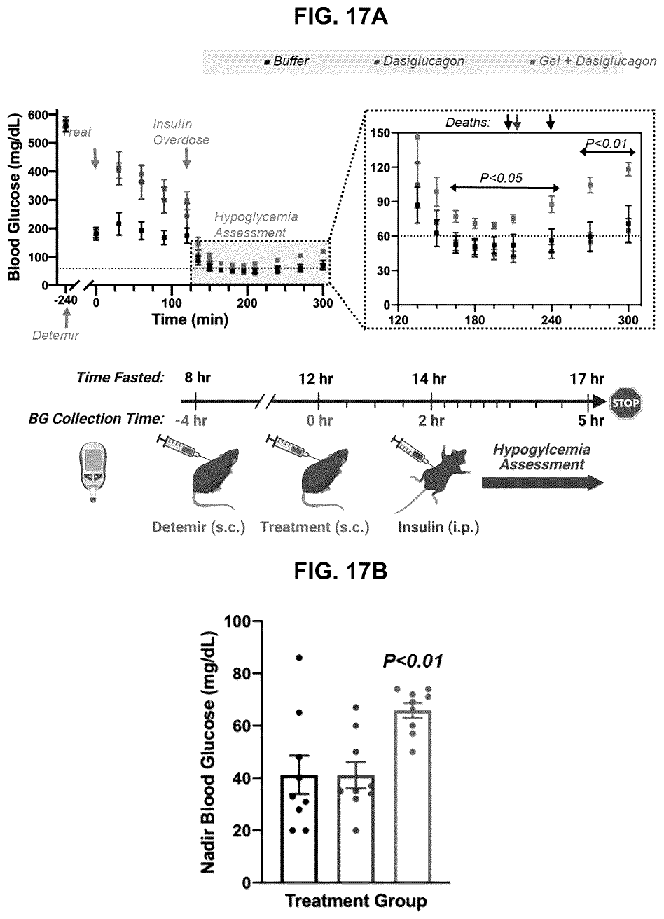

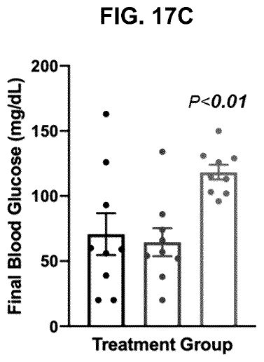

With a hydrogel material that leveraged GOₓ to achieve stability in the presence of a consumable glucose fuel, but which dissipated under conditions of reduced glucose to accelerate the release of its dasiglucagon payload, the next step was to assess the protective effect of this material to limit the onset and severity of a subsequent hypoglycemic event. For these studies, a mouse model was developed using streptozotocin (STZ) for chemically induced diabetes. STZ mice were chosen due to reports of dose-dependent increase in insulin secretion and blood glucose reduction seen for healthy mice treated with glucagon. Following onset of severe diabetes, the hydrogel technology described herein was assessed in a prophylactic capacity to limit the onset and severity of hypoglycemia upon an insulin overdose (FIG. 17A-C).

With limited reports assessing the protective use of glucose-responsive glucagon therapy, it was necessary to develop an animal model that could recreate some of the clinical features expected in using a material designed to maintain sequestered glucagon onboard in the event of a subsequent hypoglycemic episode (FIG. 17A). STZ mice were fasted to increase sensitivity to both insulin and glucagon and limit confounding data arising from variable eating patterns. After a 9 h fast, basal insulin detemir (Novo Nordisk, Levemir®) was administered to correct blood glucose to within the normoglycemic range for a healthy mouse (˜180-200 mg/dL) to simulate insulin-dependent blood glucose control in diabetes. Insulin detemir is a clinically used derivative that leverages serum albumin binding through its modification with a saturated alkyl chain to achieve long-lasting basal function, offering a duration of action of ˜18-20 h in humans. The intention of this step was to achieve a consistent blood glucose level in mice on a plane of normoglycemia, an outcome evident in data for all mice reported in this study (184±49 mg/dL, mean±std dev, n=27). Treatments (n=9/group) consisting of a sham (buffer) control, a dose of 0.01 mg dasiglucagon, or 1% w/v hydrogel containing 0.01 mg dasiglucagon along with GOₓ/catalase were next administered subcutaneously. Following 2 h, an insulin challenge was performed by injection of recombinant human insulin to induce hypoglycemia. Blood glucose was monitored serially throughout the process.

Following treatment with dasiglucagon alone or loaded within the hydrogel, blood glucose levels sharply increased relative to the buffer control at 30 min (FIG. 17A). This finding indicates active signaling of dasiglucagon in both treatments. Such a rapid peak is consistent with the known time to action of dasiglucagon in clinical trials. Unfortunately, this result points to leakage of active dasiglucagon upon administration of hydrogels under normoglycemic conditions; this is to be expected based on the near-instant release of ˜20% of the dasiglucagon payload for release assays at 200 mg/dL. Future iterations of glucose-fueled hydrogels for dasiglucagon delivery, particularly if intended as prophylactic rescue devices, must address this initial burst release so as not to interfere with the process of blood glucose control in insulin-centered therapeutic management.

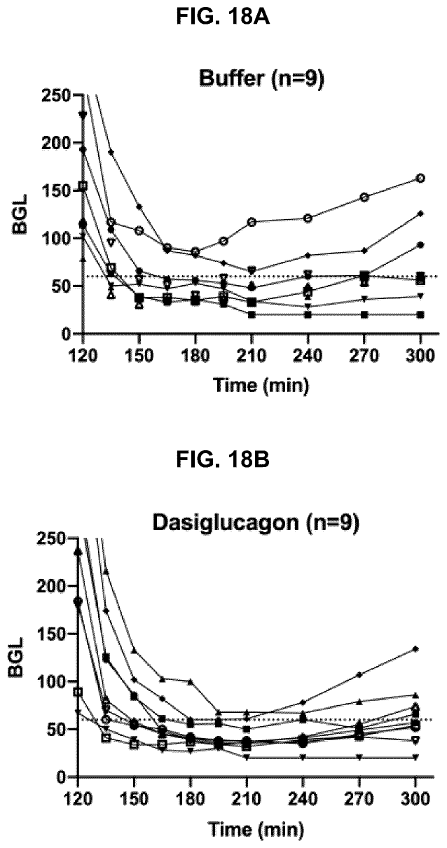

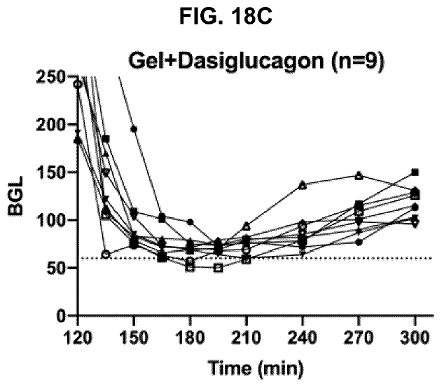

Hypoglycemia was induced 2 h after prophylactic glucagon treatment by intraperitoneal administration of recombinant insulin and blood glucose was monitored to track the onset and severity of hypoglycemia (FIG. 17A, FIG. 18A-C). The average blood glucose for mice treated with dasiglucagon-loaded hydrogels exceeded that for the other two treatments, achieving significantly (p<0.05) higher blood glucose at 45 min following insulin challenge through the end of the study. The extent of hypoglycemia can be visualized by the minimum (nadir) blood glucose value measured for each group (FIG. 17B). By this metric, the nanofibrillar hydrogel treatment (65.9±2.8 mg/dL) demonstrated significantly (p<0.01) less hypoglycemia than treatment with either dasiglucagon alone (41.1±5.0 mg/dL) or the buffer control (41.2±7.3 mg/dL). Not only did the nanofibrillar hydrogel delivery limit the onset and extent of hypoglycemia, but it demonstrated protection compared to limited observations of death for buffer (2 of 9) and dasiglucagon (1 of 9) treatments (FIG. 17A).

To assess recovery from hypoglycemia, blood glucose levels were compared at the endpoint of the study, 3 h following insulin challenge, and 5 h following administration of prophylactic treatment. By this metric, the nanofibrillar hydrogel treatment (118.3±5.7 mg/dL) demonstrated significantly (p<0.01) faster recovery than treatment with either dasiglucagon alone (65.6±10.8 mg/dL) or the buffer control (70.7±16.1 mg/dL) (FIG. 17C). Taken together with data showing a reduced severity of hypoglycemia, these data demonstrate that the nanofibrillar hydrogel achieves more rapid recovery following induction of hypoglycemia.

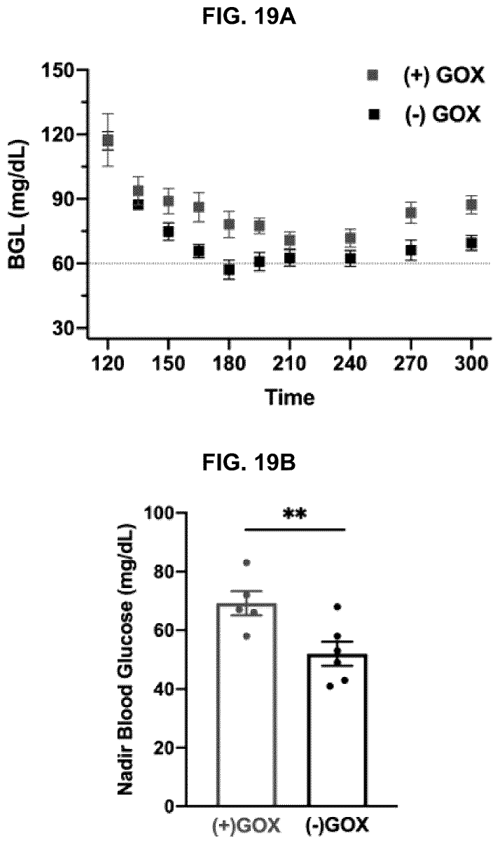

The fully formulated 50 μL hydrogel was located through necropsy in the subcutaneous space for up to 5 days following its administration in healthy mice, but could not be located at 7 d. As such, the material has reasonable stability over multiple days following administration, even when applied to a healthy mouse at a normoglycemic state. Yet the stability over time under normoglycemic conditions still lags behind related PA-based materials that form stable hydrogels at neutral pH, which remain in the subcutaneous tissue for over 30 d. Preliminary experiments also supported a functional role for GOₓ in actuating blood glucose level to dictate dasiglucagon release from the hydrogel in vivo, supporting glucose-dictated release as opposed to simple controlled release as the mechanism of hydrogel function in protecting against hypoglycemia (FIG. 19A-B).

The limited prior studies evaluating glucagon release triggered by insulin overdose and hypoglycemia makes contextualizing the present findings a challenge. Microneedle arrays have been most commonly explored in this area, leveraging insulin-binding aptamers or glucose-binding phenylboronic acid polymers to trigger transcutaneous release of encapsulated glucagon. Compared to these works, the current model may be more clinically relevant in its use of basal insulin detemir to begin studies from a point of glucose control instead of from a hyperglycemic (e.g., 400+ mg/dL) state. The use of fasting, and the protocols in cases where it has been previously used, also vary across these prior studies. Nonetheless, the results presented here compare favorably. In terms of protection against hypoglycemia, 0 of 9 mice treated with the glucagon-loaded nanofibrillar hydrogel reached levels below 50 mg/dL, whereas 7 of 9 mice in the buffer-treated group fell below this same level. This extent of hypoglycemia in the control is at least as severe as that reported in other studies, validating the relevance of the present model. Compared to this same prior work, the glucagon-treated group here began to recover from hypoglycemia within 90 min of insulin challenge, whereas the blood glucose profile in these prior glucagon-releasing microneedles remained flat or slightly decreased for the full period shown following insulin overdose (2.5 h). The expected duration of action for insulin detemir in the present model also means that recovery occurs in spite of residual basal insulin action. Overall, the results presented here to use transient glucose-stabilized hydrogels for glucagon delivery demonstrate promise in the context of other works in this space.

Glucagon leakage leading to blood glucose instability under conditions of normoglycemia would not be ideal for a once-nightly prophylactic treatment to prevent sudden onset of nocturnal hypoglycemia, as in the envisioned use case here. At the same time, glucagon has been actively explored in affording better control and more effective insulin dosing, such as in clinical work exploring control from dual-hormone pumps. Thus, technologies like the current system that offer glucose-directed control over the rate of glucagon release may be integrated more broadly within an arsenal of therapeutic strategies for better and more responsive blood glucose management, working to deliver on approaches seeking a “fully synthetic pancreas.” Indeed, inspirational work in dual-hormone microneedles points to a possibility for future material designs that pair on-demand glucagon and insulin release for better blood glucose control.

The approach outlined here demonstrates the use of enzymatic actuation consuming a ubiquitous biological and disease-relevant glucose fuel to drive the formation and stability of a supramolecular nanofibrillar hydrogel. These materials afforded glucose-directed release of a therapeutic glucagon analogue in a manner inversely related to glucose concentrations, spanning a physiologically relevant range. Moreover, this approach demonstrated the capacity to limit both the extent and duration of hypoglycemia in a diabetic mouse model when administered in a prophylactic capacity in advance of an insulin challenge. Relative to the body of work in glucose-responsive materials, the majority of which respond to high glucose levels to release insulin, the present approach offers a new paradigm in material design. This observation of material stability in the presence of continuously available glucose fuel is furthermore reminiscent of work seeking to achieve non-equilibrium steady states in materials under enzymatic control. With inspiration from many functional non-equilibrium materials in the living world, routes to engineer synthetic analogues of such materials have been active areas of discovery. Whereas the vast majority of such non-equilibrium systems reported to date are highly fundamental or have leveraged non-biologically relevant fuel sources, the present work demonstrates functional utility of similarly inspired approaches to engineer materials for a therapeutic application driven by a biological and disease-relevant glucose analyte as fuel. Accordingly, this general approach to develop glucose-fueled responsive materials holds promise for further development, either as a protective approach against hypoglycemia or as a component of a fully synthetic strategy for dual-hormone blood glucose control in diabetes.

1. A composition comprising a peptide of formula (I): 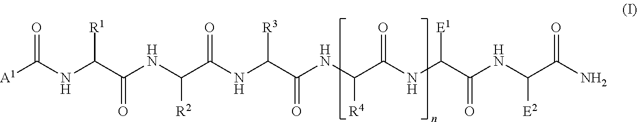
wherein:
A¹ is C₆₋₂₀alkyl;
R¹, R², R³, and R⁴, at each occurrence, are independently C₁₋₆alkyl, C₃₋₆cycloalkyl, C₁₋₂haloalkyl, C₁₋₄hydroxyalkyl, halogen, —CN, —OR¹¹, —NHR¹¹, —CO₂R¹¹, —N(R¹¹)₂, —C(O)NHR¹¹, or —C(O)N(R¹¹)₂;
R¹¹, at each occurrence, is independently hydrogen, C₁₋₄alkyl, or C₃₋₆cycloalkyl;
n is 1-3; and
E¹ and E² are each independently

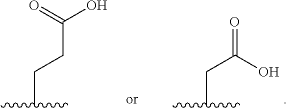 [SMILES: *CCC(=O)O ; *CC(=O)O]

2. The composition of claim 1, wherein A¹ is C₉₋₁₅alkyl.

3. The composition of claim 1, wherein A¹ is linear.

4. The composition of claim 1, wherein R¹, R², R³, and R⁴, at each occurrence, are each independently C₁₋₄alkyl.

5. The composition of claim 1, wherein R¹, R², R³, and R⁴, at each occurrence, are each independently methyl or isopropyl.

6. The composition of claim 1, wherein 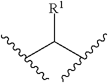
is

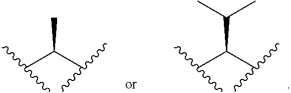

7. The composition of claim 1, wherein 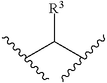
is

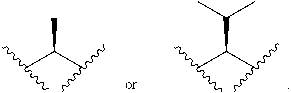

8. The composition of claim 1, wherein 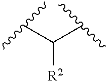
is

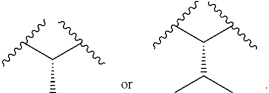

9. The composition of claim 1, wherein 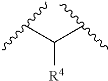
is

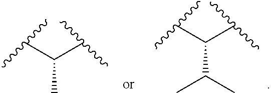

10. The composition of claim 1, wherein E¹ and E² are each

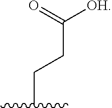 [SMILES: *CCC(=O)O]

11. The composition of claim 1, wherein 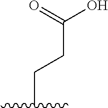 [SMILES: *CCC(=O)O]
is

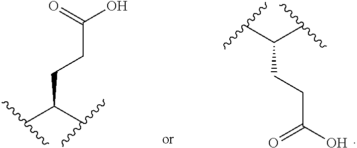

12. The composition of claim 1, wherein the peptide of formula (I) is a peptide of formula (I-a):

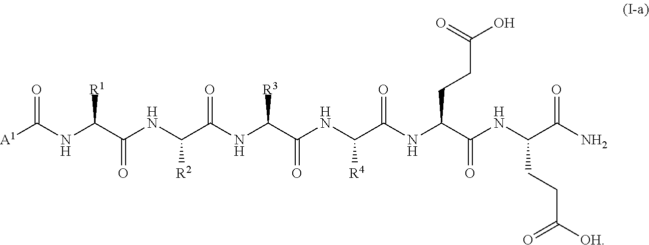

13. The composition of claim 1, wherein the peptide of formula (I) is a peptide selected from the group consisting of:

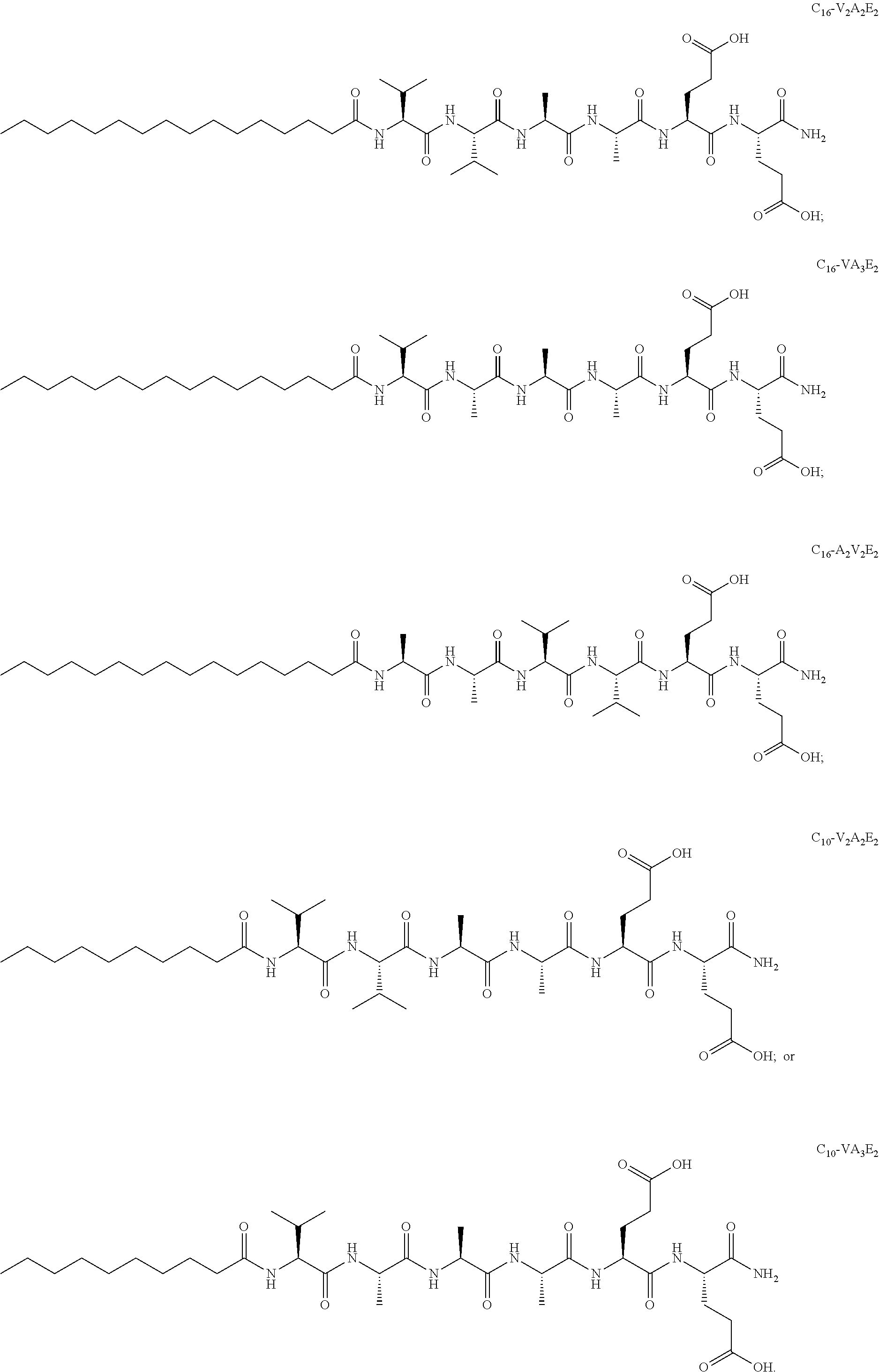

14. The composition of claim 1, wherein the peptide of formula (I) is:

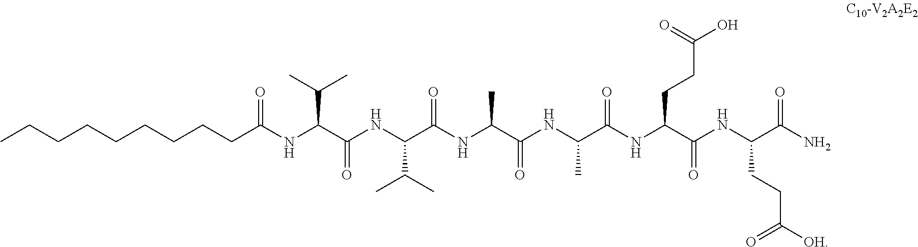 [SMILES: CCCCCCCCCC(=O)N[C@H](C(=O)N[C@H](C(=O)N[C@@H](C)C(=O)N[C@@H](C)C(=O)N[C@@H](CCC(=O)O)C(=O)N[C@@H](CCC(=O)O)C(N)=O)C(C)C)C(C)C]

15. The composition of claim 1, wherein, at a pH of about 5, the peptide of formula (I) self-assembles to form a hydrogel.

16. The composition of claim 15, wherein at a pH of about 7, the hydrogel disassembles.

17. A hydrogel comprising:
glucagon or a glucagon analogue;
a peptide of formula (I): 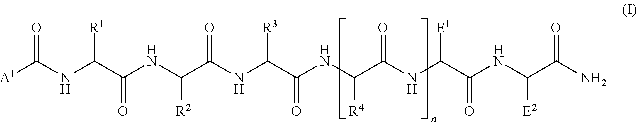
wherein:
A¹ is C₆₋₂₀alkyl;
R¹, R², R³, and R⁴, at each occurrence, are independently C₁₋₆alkyl, C₃₋₆cycloalkyl, C₁₋₂haloalkyl, C₁₋₄hydroxyalkyl, halogen, —CN, —OR¹¹, —NHR¹¹, —CO₂R¹¹, —N(R¹¹)₂, —C(O)NHR¹¹, or —C(O)N(R¹¹)₂;
R¹¹, at each occurrence, is independently hydrogen, C₁₋₄alkyl, or C₃₋₆cycloalkyl;
n is 1-3; and
E¹ and E² are each independently

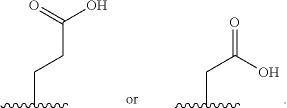 [SMILES: *CCC(=O)O ; *CC(=O)O]

18. The hydrogel of claim 17, wherein A¹ is C₉₋₁₅alkyl.

19. The hydrogel of claim 17, wherein R¹, R², R³, and R⁴, at each occurrence, are each independently methyl or isopropyl.

20. The hydrogel of claim 17, wherein the peptide of formula (I) is a peptide of formula (I-a):

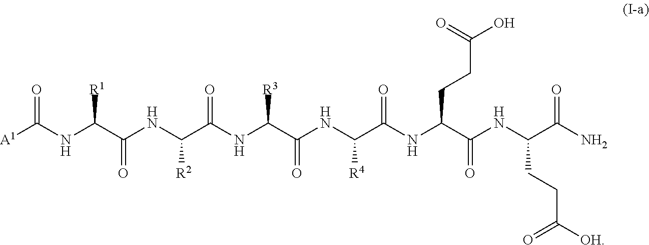

21. The hydrogel of claim 17, wherein the peptide of formula (I) is:

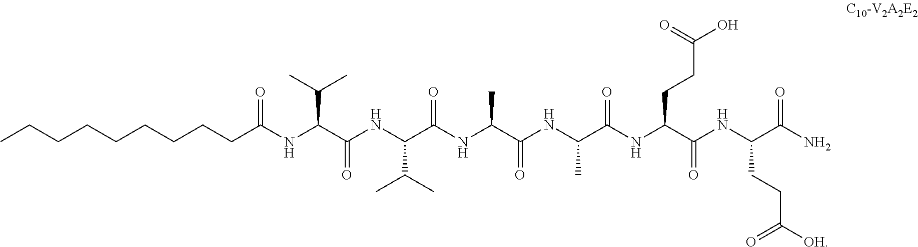 [SMILES: CCCCCCCCCC(=O)N[C@H](C(=O)N[C@H](C(=O)N[C@@H](C)C(=O)N[C@@H](C)C(=O)N[C@@H](CCC(=O)O)C(=O)N[C@@H](CCC(=O)O)C(N)=O)C(C)C)C(C)C]

22. The hydrogel of claim 17, wherein in a solution having a pH of about 5, the hydrogel is intact.

23. The hydrogel of claim 17, wherein in a solution having a pH of about 7, the hydrogel disassembles.

24. The hydrogel of claim 17, wherein the glucagon analogue comprises one or more of: dasiglucagon or a depsi-glucagon analogue.

25. A pharmaceutical composition comprising the hydrogel of claim 17, wherein the glucagon or the glucagon analogue is encapsulated within the hydrogel.

26. A method of treating an insulin disorder, the method comprising administering a therapeutically effective amount of the pharmaceutical composition of claim 25 to a subject in need thereof.

27-35. (canceled)
